(19) United States
(12) Patent Application Publication
(10) Pub. No.: US 2024/0226224 A1
(43) Pub. Date: Jul. 11, 2024

(54) COMPOSITIONS

(71) Applicant: Givaudan SA, VERNIER (CH)

(72) Inventors: Pascale Elizabeth Renée FANÇA-BERTHON, Le Thor (FR); Stéphanie CALAFAT, Scarsdale, NY (US); Cristina Maria RANZINI, Avignon (FR); Gaetan ANTONI, Morieres les Avignon (FR); Romain LECOZANNET, Montfavet (FR); Rosa NAVARRO, Santiago (CL); Yoann HEBERT, Roquemaure (FR)

(21) Appl. No.: 18/558,183

(22) PCT Filed: Apr. 28, 2022
(86) PCT No.: PCT/EP2022/061342

§ 371 Date: Oct. 30, 2023

Related U.S. Application Data
(60) Provisional application No. 63/182,551, filed on Apr. 30, 2021.

(51) Int. Cl. A61K 36/9068; A61K 31/12

(52) U.S. Cl. CPC A61K 36/9068; A61K 31/12

(57) ABSTRACT

The present invention relates to compositions comprising gingeroids or ginger oleoresin; in particular, compositions comprising gingeroids or ginger oleoresin that are highly water soluble and have an increased bioavailability. The present invention also relates to processes for providing such compositions and uses of such compositions.

### FIELD OF THE INVENTION

The present invention relates to compositions comprising ginger or; in particular, compositions comprising ginger oleoresin that are highly water insoluble. Gingeroids are defined as the sum of gingerols, including but not limited to 6-gingerol, 8-gingerol and 10-gingerol, and shogaols, including but not limited to 6-shogaol, 8-shogaol and 10-shogaol. The present invention also relates to processes for providing such compositions and uses of such compositions.

### BACKGROUND OF THE INVENTION

The listing or discussion of an apparently prior-published document in this specification should not necessarily be taken as an acknowledgement that the document is part of the state of the art or is common general knowledge.

Ginger, the rhizome of Zingiber officinale, is a member of the Zingberaceae family that has been used as a spice for many years. Ginger contains a wide variety of volatile and non-volatile compounds in varying concentrations depending on where the plant has been cultivated, and the methods used to harvest and process the plant. The major constituents in ginger comprise carbohydrates, lipids, terpenes and phenolic compounds. Phenolic compounds include gingerol, paradols and shogaol.

The rhizomes have been used since antiquity in the various traditional systems of medicine to treat cold, fever, sore throats, infectious diseases, arthritis, rheumatism, sprains, muscular aches, pains, cramps, hypertension, dementia, migraine, nervous diseases, gingivitis, toothache, asthma, stroke, and diabetes and also used as home remedy in treating various gastric ailments like constipation, diarrhea, dyspepsia, belching, bloating, gastritis, epigastric discomfort, gastric ulcerations, indigestion, nausea, and vomiting (Giacosa, A., et al. (2015). Eur Rev Med Pharmacol Sci, 19(7), 1291-6.; Haniadka, R., et al. (2013). Food & function, 4(6), 845-855.; Lete, I., & Allue, J. (2016). The Effectiveness Of Ginger In The Prevention Of Nausea And Vomiting During Pregnancy And Chemotherapy (jurnal). Spain: Clinical Management Unit of Obstetrics and Gynecology, Hospital Universitario Araba, Vitoria, Spain. Plant Physiology Laboratory, Faculty of Biosciences, Universitat Autónoma de Barcelona, Bellatera, Spain).

This long and established history of medicinal use of ginger in humans, coupled with the increased interest in natural remedies, has stimulated clinical trials to scientifically assess the effectiveness of ginger as an adjuvant therapy or as a complementary and alternative medicine in a number of diseases especially gastrointestinal ailments (Lete, I., & Allué, J. (2016). The Effectiveness of Ginger In The Prevention of Nausea And Vomiting During Pregnancy And Chemotherapy (jurnal). Spain: Clinical Management Unit of Obstetrics and Gynecology, Hospital Universitario Araba, Vitoria, Spain. Plant Physiology Laboratory, Faculty of Biosciences, Universitat Autónoma de Barcelona, Bellatera, Spain. Lete & Allué, 2016).

However, although ginger has shown efficacy against numerous human disorders, it is also known to have limited bioavailability due to poor absorption, rapid metabolism, and rapid systemic elimination. Ginger extracts highly enriched in gingeroids such as oleoresins were found to be poorly soluble in water.

The rapid metabolism, poor water solubility, and poor uptake by tissues drastically limits the potential utility of ginger, including the potential use of gingeroids (as used herein to refer to both gingerols and shogaols, in particular 6, 8 and 10 gingerol and 6, 8, and 10 shogaol) in the treatment of conditions such as gastrointestinal disorders.

The present invention seeks to address the abovementioned problems associated with ginger's poor solubility in water and provide a composition comprising ginger/gingeroids that is both highly soluble in water and stable at physiological pH.

### DISCLOSURE OF THE INVENTION

The present inventors have surprisingly found that a composition comprising ginger oleoresin or gingeroids, a gum (such as gum Arabic) and at least one saponin (such as an extract obtained from or obtainable from quillaja), provides a slow release of the gingeroids, is highly water soluble and stable at physiological pH and has an increased bioaccessibility, bioavailability, bio-efficacy, and/or bioactivity of said gingeroids.

According to the present invention, there is provided a composition comprising:
(i) gingeroids;
(ii) gum Arabic, guar gum, xantham gum, locust bean gum, gum tragacanth or mixtures thereof; and
(iii) at least one saponin.

Also, according to the present invention, there is provided a slow release composition comprising:
(i) gingeroids;
(ii) gum Arabic, guar gum, xantham gum, locust bean gum, gum tragacanth or mixtures thereof; and
(III) at least one saponin.

Such compositions may be referred to hereinafter as the “compositions of the invention”. According to the present invention, with the term “slow-release” is meant a delayed- and extended-release. This means that the active principle (gingeroids) are released after at least 30 minutes and then over a prolonged period of time of at least 1, 4 or at least 8 hours. The inventors of the present invention have shown that in the present compositions of the invention, the actives (gingeroids) are released much slower that in the control ginger extracts. The slow release effect is shown on table 11 and 13 where it is seen similar maximum gingeroids level in blood at a similar Tmax between formulation while the composition of the invention provides a slower decrease in blood gingeroids concentration in any time points: 30 min, 1, 2, 4 and 8 hours compared with both Ginger powder and Ginger 5%. This leads to a half-life increase of 2.84 fold of the composition of the invention versus the ginger Powder and a half-life increase on of 1.8 of the composition of the invention versus the ginger 5%. The gingeroids used in the compositions of the invention may be obtained from any source. However, it is preferred that the gingeroids are obtained from a natural source, i.e. the gingeroids are not synthetic, but are plant based.

Gingeroids are defined in the present invention as the sum of gingerols, including but limited to 6-gingerol, 8-gingerol and 10-gingerol and shogaols including but not limited to 6-shogaol, 8-shogaol and 10-shogaol. Other gingerols and shogaols may be present in the composition of the invention such as 12-gingerol.

The compositions may comprise at least about 5% gingeroids, such as at least about 7.5%, or at least about 10% or at least about 20% gingeroids by weight of the composition.

For example, the gingeroids may be present in an amount from about 5% to about 60%, such as from about 7.5% to about 50%, or from about 10% to about 40% by weight of the composition.

The gingeroids may be obtained or obtainable from the root (rhizome) of ginger (Zingiber officinale). The gingeroids may be provided by extraction and optionally purification from the root (rhizome) of ginger (Zingiber officinale). Those ginger extracts in the present invention includes, without limitation, ginger oleoresin, defatted ginger oleoresin and mixtures thereof. Thus, the gingeroids may be in the form of an extract or purified extract of the root (rhizome) of ginger (Zingiber officinale) (such as a ginger oleoresin, defatted ginger oleoresin and mixtures thereof), wherein the extract comprises from about 10% to about 100% gingeroids, such as from about 10%, 15%, 20%, 25%, 30%, 35%, 40%, 45%, 50%, 55%, 60%, 65%, 70%, 75%, 80%, 85% or 90% to about 95%, 90%, 85%, 80%, 75%, 70%, 65%, 60%, 55%, 50% or 45% gingeroids by weight of the extract.

Where the gingeroids are provided as an extract of the root (rhizome) of ginger (Zingiber officinale) (such as ginger oleoresin, defatted ginger oleoresin and mixtures thereof) the root (rhizome) of ginger (Zingiber officinale), ginger oleoresin, defatted ginger oleoresin and mixtures thereof may be extracted using an organic solvent or a non-organic solvent or mixtures thereof.

For example, the root (rhizome) of ginger (Zingiber officinale) may be subjected to extraction with an organic solvent, at one or more temperatures ranging from 0 to 150° C., depending on the solvent used. The organic solvent extract may be separated from the remaining root (rhizome) of ginger grounds by any method known in the art. The organic extract may be optionally concentrated using any suitable method known in the art.

Examples of suitable organic solvents include, but are not limited to, hydrocarbons, such as pentane, petroleum ether, hexane, heptane or cyclohexane; alcohols such as methanol, 2-methoxyethanol, ethanol, n-propanol, isopropanol, all isomers of butyl alcohol, benzyl alcohol, 1,2,6-trihydroxyhexane, ethylene glycol, 1,2-propanediol, dipropylene glycol, 1,3-butanediol, 2-butoxyethanol, 1,3-butylene glycol, glycerol; esters, such as ethyl acetate, iso-propyl acetate, 2-butoxyethyl acetate, glycerol esters such as glyceryl diacetate, glyceryl triacetate, glyceryl tributyrate; ethers, such as diethyl ether, 2-ethoxyethanol; ketones, such as acetone, 2-butanone; betaine, toluene, chlorinated or fluorinated hydrocarbons such as dichloromethane; and mixtures thereof, optionally with water, including eutectic mixtures. In a preferred embodiment, the organic solvent is ethyl acetate.

For example, the root (rhizome) of ginger (Zingiber officinale) may be subjected to extraction with an alcoholic solvent or a hydro-alcoholic solvent, at one or more temperatures ranging from 0 to 150° C., depending on the solvent used. For example, the alcohol-based extraction solvent may be water/methanol (i.e. a mixture of water and methanol) or water/ethanol (i.e. a mixture of water and ethanol) or methanol or ethanol The organic solvent extract may be separated from the remaining root (rhizome) of ginger grounds by any method known in the art. The alcoholic or an hydro-alcoholic extract may be optionally concentrated using any suitable method known in the art.

Where the extraction solvent comprises a water/alcohol mixture the ratio of water to alcohol may be from about 25:75 to about 1:99, such as from about 20:80 to about 5:95 or about 10:90. For example, the extraction solvent may be water/ethanol in a ratio of from about 25:75 to about 1:99, such as from about 20:80 to about 5:95 or about 10:90.

Alternatively, the root (rhizome) of ginger (Zingiber officinale) may be extracted using CO2 extraction.

The extract (such as an organic extract or CO2 extract) may then be further purified to provide a purified extract of gingeroids comprising from about 10% to about 100% gingeroids, such as from about 10%, 15%, 20%, 25%, 30%, 35%, 40%, 45%, 50%, 55%, 60%, 65%, 70%, 75%, 80%, 85% or 90% to about 95%, 90%, 85%, 80%, 75%, 70%, 65%, 60%, 55%, 50% or 45% gingeroids by weight of the extract.

The purification of the extract may be performed using such techniques known in the art. Typically, the extract is purified using an alcohol-based solvent, such as 100% methanol or 100% ethanol.

The extract may optionally be dried to remove any excess solvent.

The gingeroids may be provided as a liquid or a powder, such as a powder. For example, a powdered ginger extract. In a preferred embodiment, the gingeroids are provided as a oleoresin.

As noted above, as used herein, the term “gingeroids” includes but is not limited to 6, 8 and 10 gingerol and 6, 8, and 10 shogaol.

Where the gingeroids are provided in the form of an extract from the root (rhizome) of ginger (Zingiber officinale) (such as ginger oleoresin, defatted ginger oleoresin and mixtures thereof) as previously defined, the composition may comprise the extract in an amount of at least 10% by weight of the composition, such as at least 15%, at least 20% at least 30% or at least 40%, or at least 50% by weight of the composition.

For example, the composition may comprise the extract from the root (rhizome) of ginger (Zingiber officinale) (such as a ginger oleoresin, defatted ginger oleoresin and mixtures thereof) as previously defined in an amount of from about 10% to about 40% by weight of the composition, such as from about 15% to about 30% by weight of the composition.

For example, the composition may comprise from about 25% (i.e. 30%) to about 35% of the extract from the root (rhizome) of ginger (Zingiber officinale) (such as ginger oleoresin, defatted ginger oleoresin and mixtures thereof) by weight of the composition, where the extract from the root (rhizome) of ginger (Zingiber officinale), (such as ginger oleoresin, defatted ginger oleoresin and mixtures thereof) comprises from about 5%, 10%, 20%, 30%, 40% to about 50%, 60%, 70%, 80% or about 99%, such as from about 10% to 20%, such as 11% of gingeroids by weight of the extract from the root (rhizome) of ginger (Zingiber officinale) (such as ginger oleoresin, defatted ginger oleoresin and mixtures thereof), providing a composition that comprises from about 5% to 30% gingeroids by weight of the composition.

It may be preferred that the gingeroids are provided in the form of ginger oleoresin.

Thus, the present invention also provides a composition comprising:
(i) ginger oleoresin;
(ii) gum Arabic, guar gum, xantham gum, locust bean gum, gum tragacanth or mixtures thereof; and
(iii) at least one saponin. In a preferred embodiment, the gum is gum Arabic.

The ginger oleoresin may be obtained via super critical CO2 extraction of ginger roots.

In certain embodiments, where the composition comprises ginger oleoresin, the ginger oleoresin may be present in the composition in an amount of at least 10% by weight of the composition, such as at least 15%, at least 20% at least 30% or at least 40%, or at least 50% by weight of the composition. For example, the composition may comprise ginger oleoresin in an amount of from about 10% to about 40% by weight of the composition, such as from about 15% to about 30% by weight of the composition.

In certain embodiments, the final composition comprises at least 5% of gingeroids by weight of the composition, such as at least 10%, at least 15%, at least 20% at least 30% or at least 40%, or at least 50% gingeroids by weight of the composition.

For example, the composition may comprise from about 25% (i.e. 30%) to about 35% of ginger oleoresin by weight of the composition, where the ginger oleoresin comprises from about 11% of gingeroids by weight of the ginger oleoresin, providing a composition that comprises from about 5% to about 30% of gingeroids by weight of the composition.

Actually commercialized ginger oleoresins are liquid products that have a high content of lipids and fatty acids. During the extraction of gingeroids from ginger, and due to the chemical characteristics of the gingeroids, the extract that are rich in gingeroids are also rich in lipids. Other natural components, like curcumin extracts from turmeric rich in curcuminoids are in form of powders that have low or very low quantities of fats. Those curcuminoids normally are present in the form of crystals.

In certain embodiments, the ginger oleoresin, the defatted oleoresin and the mixtures thereof, has a lipid content of at least 40%, such as of at least 50%, such as of at least 70%. In certain embodiments, the ginger oleoresin has a lipid content of at least 40%, such as of at least 50%, such as of at least 70%.

In certain embodiments, the gingeroids are provided in the form of extracts such as an oleoresin and do not form crystals.

Thus, the present invention also provides a composition comprising:
(i) ginger oleoresin;
(ii) gum Arabic, guar gum, xantham gum, locust bean gum, gum tragacanth or mixtures thereof, preferably gum arabic; and
(iii) at least one saponin, wherein the ginger oleoresin comprises at least 30% of gingeroids and at least 50% of lipids, such as at least 70% of lipids.

Thus, the present invention also provides a composition comprising:
(i) ginger oleoresin;
(ii) gum Arabic, guar gum, xantham gum, locust bean gum, gum tragacanth or mixtures thereof, preferably gum arabic; and
(iii) at least one saponin, wherein the ginger oleoresin comprises at least 30% of gingeroids and at least 50% of lipids, such as at least 70% of lipids, and optionally, wherein the composition is a dry composition comprising particles having a mean diameter of from 150 to 300 and a D90 of from 280 to 500.

Thus, the present invention also provides a composition comprising:
(i) ginger oleoresin;
(ii) gum Arabic, guar gum, xantham gum, locust bean gum, gum tragacanth or mixtures thereof, preferably gum arabic; and
(iii) at least one saponin, wherein the final composition comprises at least 5% of gingeroids by weight of the composition, such as at least 10%, at least 15%, at least 20% at least 30% or at least 40%, or at least 50% gingeroids by weight of the composition and,
   optionally, wherein the composition is a dry composition comprising particles having a mean diameter of from 150 to 300 and a D90 of from 280 to 500.

The gum, such as gum Arabic, guar gum, xantham gum, locust bean gum, gum tragacanth or mixtures thereof, may be present in the compositions of the invention an amount from about 20% to about 80% by weight of the composition, such as from about 50% to about 70% by weight of the composition or about 68% by weight of the composition. In certain preferred embodiments, the gum (such as gum Arabic) may be present in the compositions of the invention in an amount from about 20% to about 80% by weight of the composition, such as from about 50% to about 70% by weight of the composition or about 68% by weight of the composition.

Natural gums as defined before are normally commercially available and are highly purified. In certain embodiments, the gum (such as gum Arabic, guar gum, xantham gum, locust bean gum, gum tragacanth or mixtures thereof) is food grade.

In a preferred embodiment, the gum is gum Arabic.

According to the present invention, there is provided a composition comprising:
(i) gingeroids;
(ii) gum Arabic, guar gum, xantham gum, locust bean gum, gum tragacanth or mixtures thereof; and
(iii) at least one saponin, wherein the composition comprises at least 5% of gingeroids by weight of the composition, such as at least 10%, at least 15%, at least 20% at least 30% or at least 40%, or at least 50% gingeroids by weight of the composition, wherein the gum (such as gum Arabic) is present in an amount from about 20% to about 80% by weight of the composition, such as from about 50% to about 70% by weight of the composition or about 68% by weight of the composition,
and optionally wherein the composition is a dry composition comprising particles having a mean diameter of from 150 to 300 and a D90 of from 280 to 500.

Thus, the present invention also provides a composition comprising:

(i) ginger oleoresin comprising gingeroids;

(ii) gum Arabic, guar gum, xantham gum, locust bean gum, gum tragacanth or mixtures thereof, preferably gum arabic; and

(iii) at least one saponin,
wherein the composition comprises at least 5% of gingeroids by weight of the composition, such as at least 10%, at least 15%, at least 20% at least 30% or at least 40%, or at least 50% gingeroids by weight of the composition,
wherein the gum (such as gum Arabic) is present in an amount from about 20% to about 80% by weight of the composition, such as from about 50% to about 70% by weight of the composition or about 68% by weight of the composition,
and optionally wherein the composition is a dry composition comprising particles having a mean diameter of from 150 to 300 and a D90 of from 280 to 500.

Saponins are a group of naturally occurring glycosides, predominantly found in the plant kingdom. They comprise a non-carbohydrate aglycone coupled to sugar chain units. Saponins are divided in two groups: steroidal and triterpene saponins. Over 100 steroidal and an even higher number of triterpene saponins have been so far identified. (K. Hostettmann, & A. Marston, Saponins (Cambridge University Press 1995). The saponin of the present invention can be of natural origin or of synthetic origin. It may be one or more saponins from the same or different origin.

For example, the saponin(s) can be obtained or obtainable from plants such as soya, beans, peas, oat, Solanum and Allium species, tomato, asparagus, tea, peanut, spinach, sugar beet, yam, blackberry, liquorice root, primula root, senega root, tea, licorice, ginseng, Quillaja (such as Quillaja saponaria), Yucca (such as Yucca shidigera), and/or Gyposphila. In one embodiment the saponin is quillaja saponin(s). In one preferred embodiment, the saponin is not a ginger saponin.

The one or more saponins used in the present invention can be highly purified or may be a natural extract.

As used herein, the term “quillaja saponin(s)”, “yucca saponi(s), “oat saponin(s)” etc, means one or more saponins that can be obtained or obtainable from any of the mem-bers of the quillaja family, or the yucca family or any of the plants that contains saponins such as the ones described before. The quillaja saponin or the mixture of quillaja saponins (or the yucca saponin or the mixture of yucca saponins) can be of synthetic or natural origin.

As will be appreciated by the person skilled in the art, as used herein the term “obtainable from” means that the saponin(s) may be obtained from a plant or may be isolated from the plant, or may be obtained from an alternative source, for example by chemical synthesis or enzymatic production. Whereas the term “obtained” as used herein, means that the sapo-nin(s) is directly derived from the plant. For example, in one embodiment, the saponin(s) can be a “natural extract comprising saponin(s)”.

The at least one saponin, may be of natural or synthetic origin.

A “purified saponin(s)” means one or more saponins of natural or synthetic origin that have a concentration of at least about 80%, at least about 90%, at least about 95%, at least about 99%, at least about 99.9% of one or more saponins as described before (such as quillaja saponin(s) and/or yucca saponin(s)).

A “saponin(s) comprising extract” means any natural extract comprising at least one type of saponin as described before that may be derived from, e.g., but not limited to soya, beans, peas, oat, Solanum and Allium species, tomato, asparagus, tea, peanut, spinach, sugar beet, yam, blackberry, liquorice root, primula root, senega root, Quillaja (such as Quillaja saponaria), Yucca (such as Yucca shidigera), and/or Gyposphila.

According to the present invention, the at least one saponin may be derived from a single source or from multiple sources.

According to the present invention, the at least one saponin comprising extract may be derived from a single source or from multiple sources.

Examples of yucca include, but are not limited to, Yucca aloifolia, Yucca angustissima, Yucca arkansana, Yucca baccata, Yucca baileyi, Yucca brevifolia, Yucca campestris, Yucca capensis, Yucca carnerosana, Yucca cernua, Yucca coahuilensis, Yucca constricta, Yucca decipiens, Yucca declinata, Yucca desmetiana, Yucca data, Yucca endlichiana, Yucca faxoniana, Yucca filamentosa, Yucca filifera, Yucca flaccida, Yucca gigantean, Yucca glauca, Yucca gloriosa, Yucca grandiflora, Yucca harrimaniae, Yucca intermedia, Yucca jaliscensis, Yucca lacandonica, Yucca linearifolia, Yucca luminosa, Yucca madrensis, Yucca mixtecana, Yucca necopina, Yucca neomexicana, Yucca pallida, Yucca periculosa, Yucca potosina, Yucca queretaroensis, Yucca reverchonii, Yucca rostrata, Yucca rupicola, Yucca schidigera, Yucca schottii, Yucca sterilis, Yucca tenuistyla, Yucca thompsoniana, Yucca treculeana, Yucca utahensis, or Yucca valida. In certain preferred embodiments the yucca is Yucca schidigera.

The most abundant Y. schidigera stem/bark saponins are steroidal saponins. They differ in the structure of their aglycon, according to which, they are classified into spirostane- or furostane-type derivatives. Primary saponins are glycosides of three C-25 epimeric pairs of sapogenins: sarsapogenin and smilagenin, markogenin and samogenin, gitogenin and neogotogenin. In both, spirostane and furostane derivatives, the C-3 carbohydrate chains are typically branched oligosaccharides with pentapyranosyl and/or hexopyranosyl units. As far as furostane bidesmosides are concerned, C-26 linked carbohydrate usually corresponds to a hexopyranose. It should be noted that derivatives of other sapogenins occur as minor compounds within Y. schidigera stem/bark.

Examples of quillaja include, but are not limited to, Quillaja brasiliensis, Quillaja lanceolata, Quillaja lancifolia, Quillaja molinae, Quillaja petiolaris, Quillaja poeppigii, Quillaja saponaria, Quillaja sellowiana, or Quillaja smegmadermos. In certain preferred embodiments the quillaja is Quillaja saponaria.

Classical methods presently used in measurement of steroidal saponins include spectro-photometric measurements, foam height measurements or gravimetric method. HPLC/ELSD technique is an accurate and reliable method that yields in results of appropriate repeatability and reproducibility.

A person of ordinary skill in the art will appreciate that, as used herein, a plant name may refer to the plant as a whole, or to any part of the plant, such as the roots, stem or trunk, bark, leaves, flower, flower stems, or seeds or a combination thereof. These plant parts may be used fresh, or dried, and may be whole, pulverized, mashed, comminuted or ground up. Extracts from any part or parts of the plant are also contemplated.

Saponins extracts can be obtained using similar extraction methods as described before for the ginger extracts. In a preferred embodiment the solvent is a methanol/water (for example 70:30 v/v) and the incubation time is of 24 hours at ambient temperature. Another extraction solvent can be only water. In a particular embodiment the quillaja containing material is incubated with water at a temperature from 50 to 100° C. (such as 50-60° C. or 100° C.). The extraction may be performed using a soxhlet apparatus or by maceration and filtration. The incubation time may be from some hours (like 10 hours) to 24 hours or more.

Saponin comprising extracts may include other compounds that are not saponins such as naturally occurring glicocomponents, polyphenols, salts and sugars.

In certain embodiments, the purified saponin(s) or the “natural extract comprising sapo-nin(s)” are from Yucca schidigera and/or Quillaja saponaria. Further, the at least one saponin or the saponin-comprising extract can be chosen from steroidal and triterpene saponins, and mixtures thereof.

In a preferred embodiment, the at least one saponin (such as a purified saponin(s) or a saponin extract) is obtained or obtainable from Quillaja (such as Q. saponaria).

In another preferred embodiment, the at least one saponin (such as a purified saponin(s) or a saponin extract) is obtained or obtainable from Yucca (such as Yucca schidigera).

In another preferred embodiment, the at least one saponin (such as a purified saponin(s) or a saponin extract) is obtained or obtainable from Quillaja (such as Q. saponaria) and from Yucca (such as Yucca schidigera).

This application concerns Yucca or Quillaja genus extracts, juice or any other product comprising saponins, more precisely saponins corresponding to any Yucca or Quillaja saponin structure. Y. schidigera or Q. Saponaria extract or spray dried extract in presence of inverted sugar or any other drying support that is well known in the art may be used in this application.

In certain embodiments of the invention, the at least one saponin is a natural extract, such as a quillaja extract, tea extract, licorice extract, beet root extract, sugar beet extract, ginseng extract, oat extract, yucca extract or a mixture thereof that comprises (or consist essentially/consist of) at least 5% w/w, or at least 10% w/w, or at least 15% w/w, or at least 20% w/w, or at least 25% w/w, or at least 30% w/w, or at least 35% w/w, or at least 40% w/w, or at least 50% w/w, or at least 60% w/w, or at least 70% w/w, or at least 80% w/w, or at least 95% w/w, of saponins. In one embodiment the saponin component may be a quillaja extract with at least 60% saponins, such as at least 80% w/w, of saponins as defined before (such as triterpenic and steroidal saponins).

The at least one saponin (such as quillaja saponin(s)) may be present in the composition of the invention in an amount from about 0.1% to about 5% by weight of the composition (w/w), such as from about 0.5% to about 3% or about 2%, such as from 1.3 to about 1.5% by weight of the composition.

As will be appreciated by the person skilled in the art, as used herein the term “obtainable from” means that the extract (for example quillaja extract) may be obtained from a plant of the quillaja genus (such as Quillaja Saponaria Molina) or may be isolated from the plant of the quillaja genus, or may be obtained from an alternative source, for example by chemical synthesis or enzymatic production. Whereas the term “obtained” as used herein, means that the extract is directly derived from the plant, for example a plant of the quillaja genus.

In one embodiment, the saponin is an extract obtained or obtainable from quillaja in the compositions may comprise at least 50% saponins, such as at least 60% saponins or at least 65% saponins by weight of the quilaja extract. For example, the quillaja used in the compositions may comprise from about 50% to about 80% or from about 60% to about 75% saponins by weight of the quillaja extract.

The saponin(s) (such as an extract obtained from or obtainable from quillaja) used in the process of the invention may be in any form, such as a liquid or a solid. For example, the saponin(s) (such as the quillaja extract) may be used in the form of a solid, such as a powder.

When present in the compositions, water and/or other solvent, such as alcohol, may be added to the solid or liquid quillaja. For example, the quillaja may be present in the compositions as an aqueous solution.

According to the present invention, there is provided a composition comprising:
(i) gingeroids;
(ii) gum Arabic, guar gum, xantham gum, locust bean gum, gum tragacanth or mixtures thereof; and
(iii) at least one saponin, wherein the final composition comprises at least 5% of gingeroids by weight of the composition, such as at least 10%, at least 15%, at least 20% at least 30% or at least 40%, or at least 50% gingeroids by weight of the composition,
wherein the saponin (such as quillaja saponin(s)) is present in the composition in an amount from about 0.1% to about 5% by weight of the composition (w/w), such as from about 0.5% to about 3% or about 2%, such as from 1.3 to about 1.5% by weight of the composition and optionally, wherein the composition is a dry composition comprising particles having an mean diameter of from 150 to 300 and a D90 of from 280 to 500.

Thus, the present invention also provides a composition comprising:
(i) ginger oleoresin comprising gingeroids;
(ii) gum Arabic, guar gum, xantham gum, locust bean gum, gum tragacanth or mixtures thereof, preferably gum arabic; and
(iii) at least one saponin,
   wherein the final composition comprises at least 5% of gingeroids by weight of the composition, such as at least 10%, at least 15%, at least 20% at least 30% or at least 40%, or at least 50% gingeroids by weight of the composition,
   wherein the saponin (such as quillaja saponin(s)) is present in the composition in an amount from about 0.1% to about 5% by weight of the composition (w/w), such as from about 0.5% to about 3% or about 2%, such as from 1.3 to about 1.5% by weight of the composition and optionally, wherein the composition is a dry composition comprising particles having an mean diameter of from 150 to 300 and a D90 of from 280 to 500.

Thus, the present invention also provides a composition comprising:

(i) ginger oleoresin comprising gingeroids;

(ii) gum Arabic, guar gum, xantham gum, locust bean gum, gum tragacanth or mixtures thereof, preferably gum arabic; and

(iii) at least one saponin,

wherein the final composition comprises at least 5% of gingeroids by weight of the composition, such as at least 10%, at least 15%, at least 20% at least 30% or at least 40%, or at least 50% gingeroids by weight of the composition,
wherein the gum (such as gum arabic) is present in an amount from about 20% to about 80% by weight of the composition, such as from about 50% to about 70% by weight of the composition or about 68% by weight of the composition,
wherein the saponin (such as quillaja saponin(s)) is present in an amount from about 0.1% to about 5% by weight of the composition (w/w), such as from about 0.5% to about 3% or about 2%, such as from 1.3 to about 1.5% by weight of the composition and optionally, wherein the composition is a dry composition comprising particles having a mean diameter of from 150 to 300 and a D90 of from 280 to 500.

The compositions may optionally comprise a food acceptable alkali metal carbonate or alkali earth metal carbonate, such as calcium carbonate. The alkali metal carbonate or alkali earth metal carbonate may be present in the compositions in an amount of from about % to about 20% alkali metal carbonate or alkali earth metal carbonate, such as from about 2.5% to about 10% or about 5% by weight of the composition.

Unless otherwise stated herein, the weight percentages listed are based on the total weight of (dry/liquid) composition obtained (w/w).

The present invention also provides a process or method for the preparation of a compositions as previously defined (or process of preparation or method of preparation of the invention), wherein the process comprises the steps of:
(i) preparing an aqueous solution of gum (such as gum Arabic) and at least one saponin;
(ii) mixing the aqueous solution from (i) with gingenoids, and optionally calcium carbonate, (ii) to provide an emulsion. In a preferred embodiment, the gingeroids are provided as ginger oleoresin. In a still more preferred embodiment, the ginger oleoresin has a lipid content of at least 40%, such as of at least 50%, such as of at least 70%.

In certain embodiments, natural gums may be used as described before (gum Arabic, guar gum, xantham gum, locust bean gum, gum tragacanth or mixtures thereof). Thus, in certain embodiments, the present invention provides a process for the preparation of a compositions as previously defined, wherein the process comprises the steps of:
(i) preparing an aqueous solution of gum Arabic, guar gum, xantham gum, locust bean gum, gum tragacanth or mixtures thereof and at least one saponin;
(ii) mixing the aqueous solution from (i) with gingenoids, and optionally calcium carbonate, to provide an emulsion.

In a preferred embodiment, the gingeroids are provided as ginger oleoresin that is in form of a liquid. In a still more preferred embodiment, the ginger oleoresin has a lipid content of at least 40%, such as of at least 50%, such as of at least 70%.

In a preferred embodiment, the mixture is homogenized.

The emulsions obtained using the method of the invention, may have droplets with an mean diameter of from 0.05 to 100 micron, such as from 0.1 to 100, such as from 0.1 to 90, from 0.1 to 50, such as from 0.1 to 10, such as from 0.05 to 10, from 0.05 to 9, from 0.05 to 8, 0.05 to 6, from 0.05 to 5, from 0.05 to 3, from 0.05 to 2, such as from 0.1 to 10, from 0.1 to 9, from 0.1 to 8, 0.1 to 6, from 0.1 to 5, from 0.1 to 3, from 0.1 to 2, 0.1 to 1, such as from 0.1 to 0.2, from 0.1 to 0.3, 0.1 to 0.4, from 0.1 to 0.5, from 0.1 to 0.7, from 0.1 to 0.9, from 0.2 to 0.6, from 0.2 to 0.5, from 0.2 to 0.8, from 0.2 to 0.9, from 0.3 to 0.9, from 0.3 to 1, such as from 0.9 to 10, such as from 10 to 80, such as from 10 to 70, such as from 10 to 50, such as from 20 to 50, such as from 20 to 30 from 0.5 to 30 microns, such as from 0.9 to 10 microns. In certain embodiments, the droplets may have a mean diameter of about 0.05, 0.1, 0.2, 0.3, 0.4, 0.5, 0.6, 0.7, 0.8, 0.9, 1, 1.2, 1.3, 1.4, 1.5, 1.6, 1.7, 1.8, 1.9, 2, 3, 4, 5, 6, 7, 8, 9, 10, 15, 20, 25, 30, 35, 40 or 45 microns.

The droplet size of the emulsion form step (ii) may be also expressed as D90. D90 describes the diameter where ninety percent of the distribution has a smaller droplet size and ten percent has a larger droplet size.

The emulsions obtained using the method of the invention, may have droplets with an particle size with a D90 of from 0.05 to 500 micron, such as from 0.1 to 100, such as from 0.1 to 90, from 0.1 to 50, such as from 0.1 to 10, such as from 0.05 to 10, from 0.05 to 9, from 0.05 to 8, 0.05 to 6, from 0.05 to 5, from 0.05 to 3, from 0.05 to 2, such as from 0.1 to 10, from 0.1 to 9, from 0.1 to 8, 0.1 to 6, from 0.1 to 5, from 0.1 to 3, from 0.1 to 2, 0.1 to 1, such as from 0.1 to 0.2, from 0.1 to 0.3, 0.1 to 0.4, from 0.1 to 0.5, from 0.1 to 0.7, from 0.1 to 0.9, from 0.2 to 0.6, from 0.2 to 0.8, from 0.2 to 0.9, from 0.3 to 0.9, from 0.3 to 1, such as 0.5 to 1, such as from 0.9 to 10, such as from 10 to 80, such as from 10 to 70, such as from 10 to 50, such as from 20 to 50, such as from 20 to 30 from 0.5 to 30 microns, such as from 0.9 to 10 microns. In certain embodiments, the D90 is of about 0.05, 0.1, 0.2, 0.3, 0.4, 0.5, 0.6, 0.7, 0.8, 0.9, 1, 1.2, 1.3, 1.4, 1.5, 1.6, 1.7, 1.8, 1.9, 2, 3, 4, 5, 6, 7, 8, 9, 10, 15, 20, 25, 30, 35, 40 or 45 microns.

The process may optionally include a step of drying the product of (ii) to provide a composition comprising particles having a mean diameter of from about 5 microns to about 1000 micron, such as 100 to 500 micron, such as 200 to 450 micron.

The drying of the product may be done using standard techniques known by the person skilled in the art such as spry drying,

In certain embodiments, the dried product is free of nanoparticles or it contains less than 10% particles (number-based) with at least one dimension smaller than 500 nm.

In certain embodiments, the particles of the dried product may have a mean diameter of from about 5 micron to about 1000 micron, such as from 10, 20, 30, 40, 50, 60, 70, 80, 90, 100, 150, 200, 250, 300, 400, 450, 500, 600, 700, 800, 900 microns to about 800, 700, 600, 550, 500, 450, 400, 350, 300, 250, 200, 150, 100, 90, 80, 70, 60, 50, 40, 30, 20, 10 microns (this wording include all combinations from each of the increasing number to any one of the decreasing numbers), such as from 900 to 800 microns, from 500 to 600 microns, from 100 to 500 micron, such as from 200 to 500 micron, such as from 100 to 300 micron, such as from 100 to 250 micron, such as from 140 to 250 microns, such as from 150 to 250 microns.

The particle size may be also expressed as D90. In certain embodiments, the particles of the dried product may have an D90 of from about 5 micron to about 1000 micron, such as from 10, 20, 30, 40, 50, 60, 70, 80, 90, 100, 150, 200, 250, 300, 400, 450, 500, 600, 700, 800, 900 microns to about 800, 700, 600, 550, 500, 450, 400, 350, 300, 250, 200, 150, 100, 90, 80, 70, 60, 50, 40, 30, 20, 10 microns (this wording include all combinations from each of the increasing number to any one of the decreasing numbers), such as from 900 to 800 microns, from 500 to 600 microns, from 100 to 500 micron, such as from 200 to 500 micron, such as from 250 to 500 micron, such as from 250 to 450 micron, such as from 280 to 500 micron.

In a preferred embodiment of the process of the invention, the gingeroids are provided as ginger oleoresin. In a still more preferred embodiment, the ginger oleoresin has a lipid content of at least 40%, such as of at least 50%, such as of at least 70% and a gingeroids content of at least 10%, such as at least 17%, such as at least 30% w/w of gingeroids.

For example, the present invention may provide a process for the preparation of a compositions as previously defined, wherein the process comprises the steps of:
(i) preparing an aqueous solution of gum Arabic and at least one saponin;
(ii) mixing the aqueous solution from (i) with gingeroids (such as a ginger oleoresin), and optionally calcium carbonate, to provide an emulsion; and
(iii) drying the product of (ii) to provide a composition comprising particles having a mean diameter of from 150 to 300 and a D90 of from 280 to 500.

After drying, (such as spray drying) the particles may be blended, grounded, milled and/or compacted to provide a more uniform size.

Typically, in the process of the invention, the gingeroids may have a purity of from about 5% to about 100% by weight of the gingeroids source, i.e. the ginger extract may comprise from about 5% to about 100% gingeroids, such as from about 5% w/w, 10% w/w, 20%, 35%, 40%, 45%, 50%, 55%, 60%, 65%, 70%, 75%, 80%, 85% or 90% to about 95%, 90%, 85%, 80%, 75%, 70%, 65%, 60%, 55%, 50% or 45% w/w gingeroids based on the percentage of total weight of the extract. In a preferred embodiment, the gingeroids may have a purity of from 20% to 40%, such as of about 30% w/w. In certain embodiments, the ginger extract may have at least 5% w/w gingeroids, such as at least 10%, such as at least 20%, such as at least 30% w/w of gingeroids.

Typically, in the process of the invention, the weight concentration of gingeroids or ginger oleoresin in the aqueous solution may be from about 1% to about 95%, such as from about 5% to about 80% or from about 7% to about 40% w/w.

In the process of the invention, the aqueous gum solution (such as gum arabioc solution) may be prepared by mixing gum (such as gum arabic) with water.

The aqueous gum (such as gum Arabic) solution may have a weight concentration of gum (such as gum arabic) of from about 20% to about 80%, such as from about 40% to about 60% w/w.

Typically, the aqueous gingeroids or ginger oleoresin solution and aqueous gum arabic solution are mixed using agitation.

In the process of the invention, the saponin(s) (such as an extract obtained from or obtainable from quillaja) may be as defined previously with respect to the compositions.

The saponin(s) (such as an extract obtained from or obtainable from quillaja) used in the process of the invention may be in any form, such as a liquid or a solid. For example, the saponin(s) (such as an extract obtained from or obtainable from quillaja) may be used in the form of a solid, such as a powder.

Typically, in the process of the invention, the at least one saponin (such as quillaja saponin(s)) may be present in the final composition of the invention in an amount from about 0.1% to about 5% by weight of the composition, such as from about 0.5% to about 3% or about 2%, such as from 1.3 to about 1.5% by weight of the composition.

In the process of the invention, the saponin(s) (that may be provided as a quillaja extract) may be mixed using agitation.

Mixing the aqueous solution comprising gum arabic and saponin(s) (such as quillaja) mixed with the gingeroids (such as ginger oleoresin) as defined above provide an emulsion.

The process of the invention may optionally include a step of removing additional solvent as required in order to provide a substantially dry product, i.e. a product where at least 90%, such as at least 95% or 99% of the water present has been removed. Additionally, a pasteurisation step may be performed.

As mentioned before, the compositions of the invention may be in the form of an emulsion or the compositions may be in the form a solid, for example, in the form of a powder.

As used herein, the term “emulsion” refers to a type of lipid dispersion that is formed by combining two liquids that do not usually mix. Typically, one of the liquids will contain a dispersion of the other liquid.

Sometimes the terms “colloid” and “emulsion” are used interchangeably, but as used herein the term emulsion applies when both phases of a mixture are liquids (ginger oleoresin as first liquid and the aqueous solution of saponins and gums as the second liquid).

A colloidal solution, occasionally identified as a colloidal suspension, is a mixture wherein solid particles are regularly suspended in a fluid. In a preferred embodiment, the product of step (ii) is not a colloidal solution but an emulsion.

In certain embodiments of the compositions of the invention, where the composition is in the form of an emulsion, the droplets may have a mean diameter of from 0.05 to 100 micron, such as from 0.1 to 100, such as from 0.1 to 90, from 0.1 to 50, such as from 0.1 to 10, such as from 0.05 to 10, from 0.05 to 9, from 0.05 to 8, 0.05 to 6, from 0.05 to 5, from 0.05 to 3, from 0.05 to 2, such as from 0.1 to 10, from 0.1 to 9, from 0.1 to 8, 0.1 to 6, from 0.1 to 5, from 0.1 to 3, from 0.1 to 2, 0.1 to 1, such as from 0.1 to 0.2, from 0.1 to 0.3, 0.1 to 0.4, from 0.1 to 0.5, from 0.1 to 0.7, from 0.1 to 0.9, from 0.2 to 0.6, from 0.2 to 0.5, from 0.2 to 0.8, from 0.2 to 0.9, from 0.3 to 0.9, from 0.3 to 1, such as from 0.9 to 10, such as from 10 to 80, such as from 10 to 70, such as from 10 to 50, such as from 20 to 50, such as from 20 to 30 from 0.5 to 30 microns, such as from 0.9 to 10 microns.

In certain embodiments, the droplets may have a mean diameter of about 0.05, 0.1, 0.2, 0.3, 0.4, 0.5, 0.6, 0.7, 0.8, 0.9, 1, 1.2, 1.3, 1.4, 1.5, 1.6, 1.7, 1.8, 1.9, 2, 3, 4, 5, 6, 7, 8, 9, 10, 15, 20, 25, 30, 35, 40 or 45 microns.

The emulsions may have droplets with a D90 of from 0.05 to 500 micron, such as from 0.1 to 100, such as from 0.1 to 90, from 0.1 to 50, such as from 0.1 to 10, such as from 0.05 to 10, from 0.05 to 9, from 0.05 to 8, 0.05 to 6, from 0.05 to 5, from 0.05 to 3, from 0.05 to 2, such as from 0.1 to 10, from 0.1 to 9, from 0.1 to 8, 0.1 to 6, from 0.1 to 5, from 0.1 to 3, from 0.1 to 2, 0.1 to 1, such as from 0.1 to 0.2, from 0.1 to 0.3, 0.1 to 0.4, from 0.1 to 0.5, from 0.1 to 0.7, from 0.1 to 0.9, from 0.2 to 0.6, from 0.2 to 0.8, from 0.2 to 0.9, from 0.3 to 0.9, from 0.3 to 1, such as 0.5 to 1, such as from 0.9 to 10, such as from 10 to 80, such as from 10 to 70, such as from 10 to 50, such as from 20 to 50, such as from 20 to 30 from 0.5 to 30 microns, such as from 0.9 to 10 microns.

In certain embodiments, the D90 is of about 0.05, 0.1, 0.2, 0.3, 0.4, 0.5, 0.6, 0.7, 0.8, 0.9, 1, 1.2, 1.3, 1.4, 1.5, 1.6, 1.7, 1.8, 1.9, 2, 3, 4, 5, 6, 7, 8, 9, 10, 15, 20, 25, 30, 35, 40 or 45 microns.

As mentioned before, the emulsion may be subsequently dried so as to obtain a solid.

Where the composition is in the form of a solid, such as a powder, the composition may, for example, comprise particles having a mean diameter of from about 5 micron to about 1000 micron, such as from 10, 20, 30, 40, 50, 60, 70, 80, 90, 100, 150, 200, 250, 300, 400, 450, 500, 600, 700, 800, 900 microns to about 800, 700, 600, 550, 500, 450, 400, 350, 300, 250, 200, 150, 100, 90, 80, 70, 60, 50, 40, 30, 20, 10 microns (this wording include all combinations from each of the increasing number to any one of the decreasing numbers), such as from 900 to 800 microns, from 500 to 600 microns, from 100 to 500 micron, such as from 200 to 500 micron, such as from 100 to 300 micron, such as from 100 to 250 micron, such as from 140 to 250 microns, such as from 150 to 250 microns.

The particle size may be also expressed as D90. In certain embodiments, the particles of the dried product may have an D90 of from about 5 micron to about 1000 micron, such as from 10, 20, 30, 40, 50, 60, 70, 80, 90, 100, 150, 200, 250, 300, 400, 450, 500, 600, 700, 800, 900 microns to about 800, 700, 600, 550, 500, 450, 400, 350, 300, 250, 200, 150, 100, 90, 80, 70, 60, 50, 40, 30, 20, 10 microns (this wording include all combinations from each of the increasing number to any one of the decreasing numbers), such as from 900 to 800 microns, from 500 to 600 microns, from 100 to 500 micron, such as from 200 to 500 micron, such as from 250 to 500 micron, such as from 250 to 450 micron, such as from 280 to 500 micron.

The particles in the compositions may be in the form of micelles.

After the formation of the particles (for example, after drying, such as spray drying) the particles may be ground and/or milled (such as ball milled) to provide a more uniform size.

The size of the particles may be measured by method CQ-MO-304 (using a Mastersizer Malvern instrument). The droplet size may be measured using Mastersizer.

The compositions may be provided in a solid or liquid form, preferably a solid form, such as a powder. By solid form, it is included that the compound may be provided as an amorphous solid, or as a crystalline or part-crystalline solid.

The compositions are typically highly water soluble and/or stable at a pH of 4 or more, such as a pH from about 4 to about 7.

By the term water soluble we mean that at least about 50%, such as at least about 60%, 70%, 80%, 90% or 95% of the composition will dissolve in water at room temperature, i.e. a temperature of about 25° C.

For the avoidance of doubt, preferences, options, particular features and the like indicated for a given aspect, feature or parameter of the invention should, unless the context indicates otherwise, be regarded as having been disclosed in combination with any and all other preferences, options particular features and the like as indicated for the same or other aspects, features and parameters of the invention.

The term “about” as used herein, e.g. when referring to a measurable value (such as an amount or weight of a particular component in the reaction mixture), refers to variations of +20%, +10%, +5%, +1%, +0.5%, or, particularly, +0.1% relative to the specified amount. For example, a variation of +0.5% with regards to the percentage of a component in the compositions, means a variation of 0.5% relative to the percentage given, i.e. +0.5% of 10% would mean a variation from 9.5% to 10.5%.

According to the present invention, the compositions may be provided in the form of a nutraceutical formulation, a dietary or food product for humans or animals (such as functional food formulations, i.e. food, drink, feed or pet food or a food, drink, feed or pet food supplements), a herbicide, a nutritional supplement, a fragrance or flavouring, a pharmaceutical or veterinary formulation, an oenological or cosmetic formulation or may form a part of a nutraceutical formulation, a dietary or food product for humans or animals (such as functional food formulations, i.e. food, drink, feed or pet food or a food, drink, feed or pet food supplements), a nutritional supplement, a fragrance or flavouring, a pharmaceutical or veterinary formulation, an oenological or cosmetic formulation.

Thus, the present invention also refers to a nutraceutical formulation, a dietary or food product for humans or animals (such as functional food formulations, i.e. food, drink, feed or pet food or a food, drink, feed or pet food supplements), a nutritional supplement, a fragrance or flavouring, a pharmaceutical or veterinary formulation, an oenological or cosmetic formulation comprising a composition of the invention as previously defined.

For example, the present invention provides a nutraceutical formulation, a dietary or food product for humans or animals (such as functional food formulations, i.e. food, drink, feed or pet food or a food, drink, feed or pet food supplements), a nutritional supplement, a fragrance or flavouring, a pharmaceutical or veterinary formulation, an oenological or cosmetic formulation comprising the compositions of the invention, consisting of, or consisting essentially of (i.e. at least 90% w/w of the nutraceutical formulation, a dietary or food product for humans or animals a nutritional supplement, a fragrance or flavouring, a pharmaceutical or veterinary formulation, an oenological or cosmetic formulation is the compositions, such as at least 95%, or 99% or 99.5% is the composition of the invention).

The present invention also provides the use of the compositions of the invention in a nutraceutical formulation, a dietary or food product for humans or animals (such as functional food formulations, i.e. food, drink, feed or pet food or a food, drink, feed or pet food supplements), a nutritional supplement, a fragrance or flavouring, a pharmaceutical or veterinary formulation, an oenological or cosmetic formulation.

Where the compositions are in the form of a nutraceutical formulation, a dietary or food product for humans or animals (such as functional food formulations, i.e. food, drink, feed or pet food or a food, drink, feed or pet food supplements), a nutritional supplement, a fragrance or flavouring, a pharmaceutical or veterinary formulation, an oenological or cosmetic formulation or may form a part of a nutraceutical formulation, a dietary or food product for humans or animals (such as functional food formulations, i.e. food, drink, feed or pet food or a food, drink, feed or pet food supplements), a nutritional supplement, a fragrance or flavouring, a pharmaceutical or veterinary formulation, an oenological or cosmetic formulation, the nutraceutical formulation, a dietary or food product for humans or animals (such as functional food formulations, i.e. food, drink, feed or pet food or a food, drink, feed or pet food supplements), a nutritional supplement, a fragrance or flavouring, a pharmaceutical or veterinary formulation, an oenological or cosmetic formulation may optionally further comprise a pharmaceutically/veterinary ingredients, such as excipients or carriers or (function) food acceptable ingredients and mixtures thereof as appropriate.

As used herein, references to pharmaceutically acceptable excipients may refer to pharmaceutically acceptable adjuvants, diluents and/or carriers as known to those skilled in the art.

Food acceptable ingredients include those known in the art (including those also referred to herein as pharmaceutically acceptable excipients) and that can be natural or non-natural, i.e. their structure may occur in nature or not. In certain instances, they can originate from natural compounds and be later modified (e.g. maltodextrin).

By “pharmaceutically/nutraceutically acceptable” we mean that the additional components of the composition are sterile and pyrogen free. Such components must also be “acceptable” in the sense of being compatible with the compositions and not deleterious to the recipients thereof. Thus, “pharmaceutically acceptable” includes any compound(s) used in forming a part of the formulation that is intended to act merely as an excipient, i.e. not intended to have biological activity itself. Thus, the pharmaceutically acceptable excipient is generally safe, non-toxic, and neither biologically nor otherwise undesirable.

Where the compositions forms part of a nutraceutical formulation, a dietary or food product for humans or animals (such as functional food formulations, i.e. food, drink, feed or pet food or a food, drink, feed or pet food supplements), a nutritional supplement, a fragrance or flavouring, a pharmaceutical or veterinary formulation, an oenological or cosmetic formulation, the compositions may be present in the nutraceutical formulation, a dietary or food product for humans or animals, a nutritional supplement, a fragrance or flavouring, a pharmaceutical or veterinary formulation, an oenological or cosmetic formulation in an amount from about 1 to about 99% by weight of the nutraceutical formulation, a dietary or food product for humans or a nutritional supplement, a fragrance or flavouring, a pharmaceutical or veterinary formulation, an oenological or cosmetic formulation, such as from about 0.01%, 0.1%, 1%, 5%, 10%, 20%, 30%, 40%, 50%, 60%, 70%, 80% or 90% to about 90%, 80%, 70%, 60%, 50%, 40%, 30%, or 20% by weight of the nutraceutical formulation, a dietary or food product for humans or animals a nutritional supplement, a fragrance or flavouring, a pharmaceutical or veterinary formulation, an oenological or cosmetic formulation.

The skilled person will understand that the compositions, either where the composition is in the form of nutraceutical formulation, a dietary or food product for humans or animals (such as functional food formulations, i.e. food, drink, feed or pet food or a food, drink, feed or pet food supplements), a nutritional supplement, a fragrance or flavouring, a pharmaceutical or veterinary formulation, an oenological or cosmetic formulation or where the nutraceutical formulation, a dietary or food product for humans or animals (such as functional food formulations, i.e. food, drink, feed or pet food or a food, drink, feed or pet food supplements), a nutritional supplement, a fragrance or flavouring, a pharmaceutical or veterinary formulation, an oenological or cosmetic formulation comprises the compositions, the nutraceutical formulation, a dietary or food product for humans or animals (such as functional food formulations, i.e. food, drink, feed or pet food or a food, drink, feed or pet food supplements), a nutritional supplement, a fragrance or flavouring, a pharmaceutical or veterinary formulation, an oenological or cosmetic formulation may be administered to a patient or subject (e.g. a human or animal patient or subject) by any suitable route, such as by the oral, rectal, nasal, pulmonary, buccal, sublingual, transdermal, intracisternal, intraperitoneal, and parenteral (including subcutaneous, intramuscular, intrathecal, intravenous and intradermal) route.

In particular, compositions of the invention and nutraceutical formulations, a dietary or food product for humans or animals (such as functional food formulations, i.e. food, drink, feed or pet food or a food, drink, feed or pet food supplements), a nutritional supplement, a fragrance or flavouring, a pharmaceutical or veterinary formulation, an oenological or cosmetic formulation comprising the compositions may be administered orally. In such instances, pharmaceutical compositions according to the present invention may be specifically formulated for administration by the oral route.

Pharmaceutical or nutraceutical formulations for oral administration include solid dosage forms such as hard or soft capsules, tablets, troches, dragees, pills, lozenges, powders and granules. Where appropriate, they can be prepared with coatings such as enteric coatings, or they can be formulated so as to provide controlled release of the active ingredient, such as sustained or prolonged release, according to methods well known in the art.

Liquid dosage forms for oral administration include solutions, emulsions, aqueous or oily/oil based suspensions, syrups and elixirs.

Nutraceutical formulations, a dietary or food product for humans or animals (such as functional food formulations, i.e. food, drink, feed or pet food or a food, drink, feed or pet food supplements), a nutritional supplement, a fragrance or flavouring, a pharmaceutical or veterinary formulation, an oenological or cosmetic formulation described herein, such as those intended for oral administration, may be prepared according to methods known to those skilled in the art, such as by bringing the components of the composition into admixture.

Such nutraceutical formulations, a dietary or food product for humans or animals (such as functional food formulations, i.e. food, drink, feed or pet food or a food, drink, feed or pet food supplements), a nutritional supplement, a fragrance or flavouring, a pharmaceutical or veterinary formulation, an oenological or cosmetic formulations as described herein may contain one or more additional components selected from the group consisting of food ingredients, such as sweetening agents, flavouring agents, colouring agents and preserving agents. Tablets may contain the active ingredient(s) in admixture with non-toxic pharmaceutically acceptable excipients (or ingredients) which are suitable for the manufacture of tablets. These excipients (or ingredients) may, for example, be: inert diluents, such as calcium carbonate, sodium carbonate, lactose, calcium phosphate or sodium phosphate; granulating and disintegrating agents, for example, corn starch, maltodextrin or alginic acid; binding agents, for example, starch, gelatine or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Suitable pharmaceutical carriers include inert solid diluents or fillers, sterile aqueous solutions and various organic solvents. Examples of solid carriers are lactose, terra alba, sucrose, cyclodextrin, maltodextrin, talc, gelatine, agar, pectin, acacia, magnesium stearate, stearic acid, arabic gum, modified starch and lower alkyl ethers of cellulose. Examples of liquid carriers are syrup, peanut oil, olive oil, phospholipids, fatty acids, fatty acid amines, polyoxyethylene and water. Moreover, the carrier or diluent may include any sustained release material known in the art, such as glyceryl monostearate or glyceryl distearate, alone or mixed with a wax.

Depending on the disorder, and the patient, to be treated, as well as the route of administration, compositions of the invention may be administered at varying doses (i.e. therapeutically effective doses, as administered to a patient in need thereof). In this regard, the skilled person will appreciate that the dose administered to a mammal, particularly a human, in the context of the present invention should be sufficient to effect a therapeutic response in the mammal over a reasonable timeframe. One skilled in the art will recognize that the selection of the exact dose and formulation and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Typically, the compositions or the nutraceutical formulation, a dietary or food product for humans or animals (such as functional food formulations, i.e. food, drink, feed or pet food or a food, drink, feed or pet food supplements), a nutritional supplement, a fragrance or flavouring, a pharmaceutical or veterinary formulation, an oenological or cosmetic formulation as defined previously is administered to provide gingeroids in an amount of from about 10 mg gingeroids, about 15 mg, about 20 mg, about 30 mg, about 40 mg, about 50 mg, about 70 mg, about 100 mg, about 200 mg, about 300 mg of gingeroids. In one embodiment the compositions or the nutraceutical formulation, a dietary or food product for humans or animals (such as functional food formulations, i.e. food, drink, feed or pet food or a food, drink, feed or pet food supplements), a nutritional supplement, a fragrance or flavouring, a pharmaceutical or veterinary formulation, an oenological or cosmetic formulation as defined previously is administered to provide gingeroids in an amount of from about 5 mg/day of gingeroids to about 400 mg/day of gingeroids, such as about 5 mg/day gingeroids, about 10 mg/day gingeroids, about 15 mg/day, about 20 mg/day, about 30 mg/day, about 40 mg/day, about 50 mg/day, about 70 mg/day, about 100 mg/day, about 200 mg/day or about 300 mg/day of gingeroids.

For example, the composition or the nutraceutical formulation, a dietary or food product for humans or animals (such as functional food formulations, i.e. food, drink, feed or pet food or a food, drink, feed or pet food supplements), a nutritional supplement, a fragrance or flavouring, a pharmaceutical or veterinary formulation, an oenological or cosmetic formulation may provide gingeroids in an amount of from about 0.08 mg of gingeroids per kg body weight to about 6.5 mg of gingeroids per body weight, such as 1.5 mg of gingeroids per kg body weight, 0.2 mg of gingeroids per kg body weight, 0.5 mg of gingeroids per kg body weight, 1 mg of gingeroids per kg body weight, 2 mg of gingeroids per kg body weight, 3 mg of gingeroids per kg body weight or 5 mg of gingeroids per kg body weight. In certain embodiments the composition may provide gingeroids in an amount of from 1.6 mg of gingeroids per kg body weight to 3.4 mg of gingeroids per kg body weight.

In certain embodiments, the gingeroids per kg body weight is in a daily dose basis.

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

The high water solubility and stability of the compositions means that the compositions can be used to improve bioaccessibility, bioavailability bioefficacy and/or bioactivity of gingeroids or ginger oleoresin in mammals.

The improved bioaccessibility, bioavailability, bioefficacy and/or bioactivity of gingeroids or ginger oleoresin allows for the compositions to be used to prevent and/or treat diseases where in the past the poor aqueous solubility and stability of gingeroids or ginger oleoresin has been an issue.

Thus, the present invention provides a method for improving bioaccessibility, bioavailability bioefficacy and/or bioactivity of gingeroids or ginger oleoresin in mammals comprising the administration of said gingeroids or ginger oleoresin in the form of a compositions as previously defined.

The method may be referred to hereinafter as the “method of the invention”.

The present invention also provides the use of a composition as previously defined for improving the bioaccessibility, bioavailability bioefficacy and/or bioactivity of gingeroids or ginger oleoresin in mammals.

The use may be referred to hereinafter as the “use of the invention”.

In the method or uses described herein the improvement in bioaccessibility, bioavailability, bioefficacy and/or bioactivity of gingeroids or ginger oleoresin in mammals may be due to the composition providing improved gastrointestinal resistance of the gingeroids or ginger oleoresin and/or improved absorption of gingeroids or ginger oleoresin by intestinal cells and/or improved blood circulation.

In the methods or uses described herein the improvement in bioaccessibility, bioavailability bioefficacy and/or bioactivity of gingeroids or ginger oleoresin in mammals may be due to the composition providing improved water solubility and/or improved stability at a pH from about 4 to about 7.

Thus, the present invention provides a method for improving the water solubility and/or pH stability of gingeroids or ginger oleoresin, wherein the method comprises the administration of said gingeroids or ginger oleoresin in the form of a composition as previously defined.

The present invention also provides the use of a composition as previously defined for improving the water solubility and/or pH stability of gingeroids or ginger oleoresin.

The present invention also provides a method for providing a slow release of gingeroids in the gut of mammals comprising the administration of said gingeroids or ginger oleoresin in the form of a composition of the invention or a nutraceutical formulation, dietary or food product for humans or animals, nutritional supplement, pharmaceutical or veterinary formulation comprising the compositions of the invention.

The present invention also provides the use of a composition of the invention or a nutraceutical formulation, dietary or food product for humans or animals, nutritional supplement, pharmaceutical or veterinary formulation according to the invention for providing a slow release of gingeroids in the gut of a mammal.

In the methods or uses described herein, the gingeroids may be selected from the group consisting of 6-gingerol 8-gingerol, 10-gingerol, 6-shogaol, 8-shogaol, 10-shogaol or mixtures thereof

As noted above, the slow release and the improved bioaccessibility, bioavailability, bioefficacy and/or bioactivity of gingeroids or ginger oleoresin allows for the compositions to be used to prevent and/or treat diseases where in the past the poor aqueous solubility and stability of gingeroids or ginger oleoresin has been an issue.

All the different affections and diseased that can be prevented or treated using ginger extracts and oleoresins (Nguyen Hoang Anh et al. Nutrients 2020, 12, 157) may be treated or prevented using the compositions of the present invention. Some examples of diseases and affections are listed herein without any kind of limitation.

Thus, the present invention provides a composition as previously defined or a nutraceutical formulation, a dietary or food product for humans or animals, a nutritional supplement, a fragrance or flavouring, a pharmaceutical or veterinary formulation, an oenological or cosmetic formulation as previously defined, for use in preventing or treating gastrointestinal disorders, metabolic disorders primary dysmenorrhea, heavy menstrual bleeding, metabolic disorders, sleep disorders (such as insomnia), neuropsychiatric disorders (such as depression and anxiety), neurodegenerative disease (such as schizophrenia, Alzheimer's disease, Parkinson's disease), and/or aged-induced cognitive declined attention, alertness and/or mood.

The present invention also provides a method of preventing or treating gastrointestinal disorders, primary dysmenorrhea, heavy menstrual bleeding, metabolic disorders, sleep disorders (such as insomnia), neuropsychiatric disorders (such as depression and anxiety), neurodegenerative disease (such as schizophrenia, Alzheimer's disease, Parkinson's disease), and/or aged-induced cognitive declined attention, alertness and/or mood, wherein the method comprises the administration of a composition as previously defined or a nutraceutical formulation, a dietary or food product for humans or animals, a nutritional supplement, a fragrance or flavouring, a pharmaceutical or veterinary formulation, an oenological or cosmetic formulation as previously defined to a patient in need thereof.

The present invention also provides the use of a composition as previously defined in the manufacture of a medicament for treating or preventing gastrointestinal disorders, primary dysmenorrhea, heavy menstrual bleeding, metabolic disorders, sleep disorders (such as insomnia), neuropsychiatric disorders (such as depression and anxiety), neurodegenerative disease (such as schizophrenia, Alzheimer's disease, Parkinson's disease), and/or aged-induced cognitive declined attention, alertness and/or mood.

The present invention also provides a method for supporting gastrointestinal health in a human or animal in need thereof, the method comprising administering to the human or animal an effective amount of a composition as previously defined or a nutraceutical formulation, a dietary or food product for humans or animals, a nutritional supplement, a fragrance or flavouring, a pharmaceutical or veterinary formulation, an oenological or cosmetic formulation as previously defined.

The present invention provides a composition as previously defined, a nutraceutical formulation, a dietary or food product for humans or animals, a nutritional supplement, a fragrance or flavouring, a pharmaceutical or veterinary formulation, an oenological or cosmetic formulation as previously defined, for use in supporting gastrointestinal health.

Gastrointestinal disorders comprises, without limitation, dyspepsia and other problems related to gastric emptying and dysrhythmia, Irritable Bowel Syndrome (IBS), constipation, hemorrhoids, anal fissures, perianal abscesses, anal fistulas, perianal infections, diverticular diseases, colitis, colon polyps, diarrhea, colorectal cancer, CINV in cancer patients, nausea or vomiting (such as chemotherapy induced nausea and vomiting, nausea and vomiting because of pregnancy, postoperative nausea and vomiting, hyperemesis gravidarum or motion sickness).

In preferred embodiments of the uses and methods described therein, the gastrointestinal disease is selected from dyspepsia, Irritable Bowel Syndrome (IBS), colorectal cancer, nausea or vomiting (such as chemotherapy induced nausea and vomiting, nausea and vomiting because of pregnancy, postoperative nausea and vomiting, hyperemesis gravidarum or motion sickness).

Other diseases and affections can be treated using ginger.

Thus the present invention provides a composition as previously defined, a nutraceutical formulation, a dietary or food product for humans or animals, a nutritional supplement, a fragrance or flavouring, a pharmaceutical or veterinary formulation, an oenological or cosmetic formulation as previously defined, for use in supporting muscle health (such as reducing muscle pain, inflammation, and dysfunction induced by exercise or muscle damage delayed onset muscle soreness)

Thus, the present invention provides a composition as previously defined or a nutraceutical formulation, a dietary or food product for humans or animals, a nutritional supplement, a fragrance or flavouring, a pharmaceutical or veterinary formulation, an oenological or cosmetic formulation as previously defined, for use in preventing or treating muscle pain, inflammation, and dysfunction induced by exercise or muscle damage delayed onset muscle soreness.

The present invention also provides a method of preventing or treating muscle pain, inflammation, and dysfunction induced by exercise or muscle damage delayed onset muscle soreness, wherein the method comprises the administration of a composition as previously defined or a nutraceutical formulation, a dietary or food product for humans or animals, a nutritional supplement, a fragrance or flavouring, a pharmaceutical or veterinary formulation, an oenological or cosmetic formulation as previously defined to a patient in need thereof.

The present invention also provides the use of a composition as previously defined in the manufacture of a medicament for preventing or treating muscle pain, inflammation, and dysfunction induced by exercise or muscle damage delayed onset muscle soreness.

The present invention also provides a method for supporting muscle health in a human or animal in need thereof, the method comprising administering to the human or animal an effective amount of a composition as previously defined or a nutraceutical formulation, a dietary or food product for humans or animals, a nutritional supplement, a fragrance or flavouring, a pharmaceutical or veterinary formulation, an oenological or cosmetic formulation as previously defined.

In the methods and uses described herein, the mammal may be a human.

As used herein, the term “bioavailability” can be defined as the fraction of ingested component available at the site of action for utilization in normal physiological functions and is determined through in vivo assays (Guerra A, Etienne-Mesmin L, Livrelli V et al (2012) Relevance and challenges in modeling human gastric and small intestinal digestion. Trends Biotechnol 30:591-600). Bioavailability is the result of three main steps: digestibility and solubility of the element in the gastrointestinal tract; absorption of the element by the intestinal cells and transport into the circulation; and incorporation from the circulation to the functional entity or target (Wienk K J H, Marx J J M, Beynen A C (1999) The concept of iron bioavailability and its assessment. Eur J Nutr 38:51-75; Etcheverry P, Grusak M A, Fleige L E (2012) Application of in vitro bioaccessibility and bioavailability methods for calcium, carotenoids, folate, iron, magnesium, polyphenols, zinc, and vitamins B6, B12, D, and E. Front Physiol 3:1-21).

As used herein, the term “bioaccessibility” can be defined as the fraction of a compound that is released from its food matrix within the gastrointestinal tract and thus becomes available for intestinal absorption (typically established from in vitro procedures). It includes the sequence of events that take place during food digestion for transformation into potentially bioaccessible material but excludes absorption/assimilation through epithelial tissue and pre-systemic metabolism (both intestinal and hepatic). (Alegría A., Garcia-Llatas G., Cilla A. (2015) Static Digestion Models: General Introduction. In: Verhoeckx K. et al. (eds) The Impact of Food Bioactives on Health. Springer, Cham)

As used herein, the term “bioactivity” can be defined as how the nutrient or bioactive compound is transported and reaches the target tissue, how it interacts with biomolecules, the metabolism or biotransformation it may experience, and the generation of biomarkers and the physiological responses induced (Alegría A., Garcia-Llatas G., Cilla A. (2015) Static Digestion Models: General Introduction. In: Verhoeckx K. et al. (eds) The Impact of Food Bioactives on Health. Springer, Cham).

Gastrointestinal disorders comprise, without limitation, dyspepsia and other problems related to gastric emptying and dysrhythmia, Irritable Bowel Syndrome (IBS), colorectal cancer, CINV in cancer patients, nausea or vomiting (such as chemotherapy induced nausea and vomiting, nausea and vomiting because of pregnancy, postoperative nausea and vomiting, hyperemesis gravidarum or motion sickness).

Metabolic disorders that can be treated with the composition of the invention includes without limitation: glucose control, insulin sensitivity, type 2 diabetes mellitus, insulin resistance, obesity related metabolic disorder reduction (such as diabetes), etc.

For the avoidance of doubt, in this specification when we use the term “comprising” or “comprises” we mean that the extract or composition being described must contain the listed ingredient(s) but may optionally contain additional ingredients. When we use the term “consisting essentially of” or “consists essentially of” we mean that the extract or composition being described must contain the listed ingredient(s) and may also contain small (for example up to 5% by weight, or up to 1% or 0.1% by weight) of other ingredients provided that any additional ingredients do not affect the essential properties of the extract or composition. When we use the term “consisting of” or “consists of” we mean that the extract or composition being described must contain the listed ingredient(s) only.

It is also intended that the terms “comprise” or “comprises” or “comprising” may be replaced with “consist” or “consisting” or “consisting essentially of” throughout the application as required.

The terms “a” and “an” are defined as one or more unless expressly stated otherwise or constrained by other language herein. An element or feature proceeded by “a” or “an” may be interpreted as one of the recited element or feature, or more than one of the element or feature. For instance, a gum may be interpreted as one type of gum or as more than gums (for example gum Arabic and guar gum).

The terms “about,” “approximately,” “essentially,” “substantially,” any other version thereof, or any other similar relative term, or similar term of approximation, are defined as being close to as understood by one having ordinary skill in the art. By way of non-limiting, illustrative embodiments, these terms are defined to be within 20% of a recited value, or defined to be within 10% of a recited value, or defined to be within 5% of a recited value, or defined to be within 4% of a recited value, or defined to be within 3% of a recited value, or defined to be within 2% of a recited value, or defined to be within 1% of a recited value, or defined to be within 0.5% of a recited value, or defined to be within 0.25% of a recited value, or defined to be within 0.1% of a recited value.

It should be understood that when an amount in weight percent is described in the present disclosure, it is intended that any and every amount within the range, including the end points, is to be considered as having been expressly disclosed. For example, the disclosure of “a range of from about 1 to about 10” is to be read as indicating each and every possible number along the continuum between about 1 and about 10. It is to be understood that the inventors appreciate and understand that any and all data points within the range are to be considered to have been specified, and that the inventors have possession of the entire range and all points within the range.

For the avoidance of doubt, preferences, options, particular features and the like indicated for a given aspect, feature or parameter of the invention should, unless the context indicates otherwise, be regarded as having been disclosed in combination with any and all other preferences, options, particular features and the like as indicated for the same or other aspects, features and parameters of the invention.

The following text sets forth a broad description of numerous different embodiments of the present disclosure. The description is to be construed as exemplary only and does not describe every possible embodiment since describing every possible embodiment would be impractical, if not impossible. It will be understood that any feature, characteristic, component, composition, ingredient, product, step or methodology described herein can be deleted, combined with or substituted for, in whole or part, any other feature, characteristic, component, composition, ingredient, product, step or methodology described herein. Numerous alternative embodiments could be implemented, using either current technology or technology developed after the filing date of this patent, which would still fall within the scope of the claims. Preferred and/or optional features of the invention will now be set out. Any aspect of the invention may be combined with any other aspect of the invention unless the context demands otherwise. Any of the preferred or optional features of any aspect may be combined, singly or in combination, with any aspect of the invention, as well as with any other preferred or optional features, unless the context demands otherwise.

### BRIEF DESCRIPTION OF FIGURES

FIG. 1: Results of visual inspection of the samples.

FIG. 2: Lab* evolution

FIG. 3: General scheme of the adapted SHIME system used to study the survival of probiotics in the gastro-intestinal tract. The system is composed of 1 reactor that is used to simulate first the stomach and then the small intestinal environment, both under fasted or fed conditions. In this experiment the system is coupled with Caco-2 transport assays.

FIG. 4: Bioaccessibility of total gingeroids after passage through the upper gastro-intestinal tract (GIT). % bioaccessibility was calculated based on the theoretical final concentration of 20 or 10 μg gingeroids/mL in the apical compartment at the start (i.e. ([AP 0 h]/20 or 10)*100). Bars represent mean±SEM (n=3).

FIG. 5: Gingeroid bioavailability calculated as % transport x % bioaccessibility with % transport defined as ([BL 4 h]/20 or 10)*100, with 20 or 10 μg/mL being the theoretically added concentration of total gingeroids to the apical compartment at time point 0 h.

FIG. 6: Gingeroid bioavailability calculated as % absorption x % bioaccessibility with % absorption defined as (([BL 4 h]+[cells 4 h])/20 or 10)*100, with 20 or 10 μg/mL being the theoretically added concentration of total gingeroids to the apical compartment at time point 0 h.

FIG. 7: Microscopic view of the dried composition A.

### EXAMPLES

The present invention will be further described by reference to the following, non-limiting examples.

### Example 1—Preparation of a Composition of the Invention

The composition A is prepared as follows:

First, an aqueous solution of gum Arabic and extract of quillaja (that has >65% of saponins) was prepared.

Then said aqueous solution of gum Arabic and quillaja was mixed with a ginger oleoresin (>30% total Gingeroids and lipid content of more than 70% w/w) The % w/w of each component is in presented on Table 1.

The ingredients are mixed to create an emulsion and then dried using a spray drying equipment (Composition A).

### Example 2. Characterization of the Composition of the Invention

TABLE 1
Different compositions used in the examples

| Name | Composition | % Gingeroids |
|---|---|---|
| Ginger 5% | Ginger oleoresin 37.5% | 5.00 |
| | Maltodextrin 14.8% | |
| | Cornstarch 45% | |
| | Silica 2.7% | |
| Composition A | Ginger oleoresin 30% | 11.09 |
| | Quillaja 2% | |
| | Gum arabic 68% | |

TABLE 2
Particle size measurements of the dry powder.

| | Composition A (Batch 1) | Composition A (Batch 2) |
|---|---|---|
| D(4:3) μm | 224 | 186 |
| D(90) μm | 447 | 387 |
| D(50) μm | 155 | 139 |
| D(10) μm | 51 | 47 |

The dried product particles were measured with a Keyence VHX-700 digital microscope. Mastersizer equipment measures the particle size and particle size distribution by laser diffraction (laser light scattering) (CQ-MO-304 METHOD)

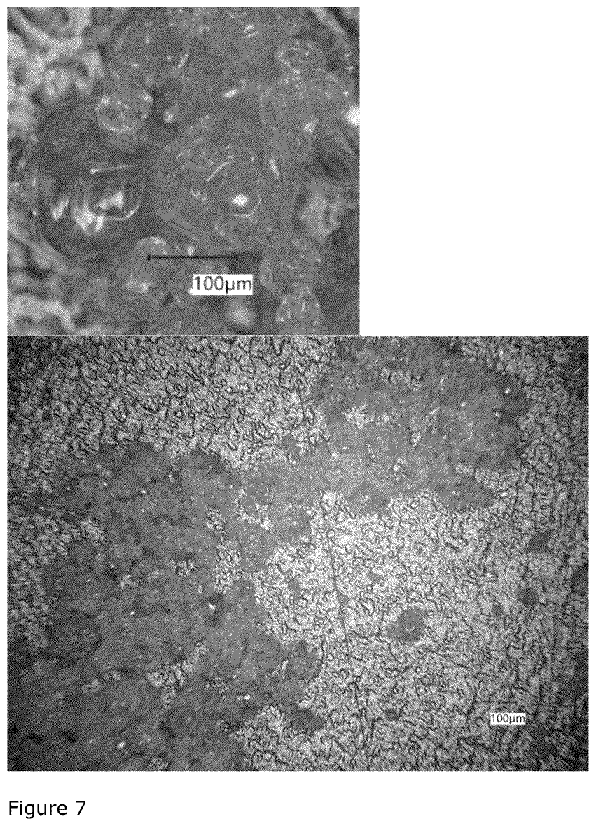

It can be seen in FIG. 7 that the Composition A particles show small spherical structures that are resulting from the dried emulsion.

### 2.1. Materials & Methods

TABLE 3
Beverage base recipe:

| Ingredient | Quantity (%) | For 60 mL (g) |
|---|---|---|
| Ginger extract | Cf table 3 | Cf table 3 |
| Water | Qsp 100% | Qsp 60 mL |
| Sucrose | 10.000 | 6.000 |
| Citric acid (50% in water distilled) | 0.376 | 0.226 |
| Potassium sorbate | 0.025 | 0.015 |
| Sodium benzoate | 0.015 | 0.009 |

Recommendation of dosage: 10 and 20 mg of gingeroids in 60 mL of beverage.

### Process Steps:

Weigh all ingredients in a glass bucket, complete with water and stir until perfect homogenization

TABLE 4
Ginger extracts composition within the RTD

| | Extract dose (mg) | Extract dose (mg) for |
|---|---|---|
| | for 60 mL RTD | 60 mL RTD |
| | Ginger 5% | Composition A |
| 10 mg gingeroids | 200 | 90.17 |
| 20 mg gingeroids | 400 | 180.34 |

TABLE 5
Stability study parameters:

| | +1 week | +2 weeks |
|---|---|---|
| T0 | Stability study: 40° C. | |
| Lab* | Lab* | Lab* |
| Visual inspection | Visual inspection | Visual inspection |

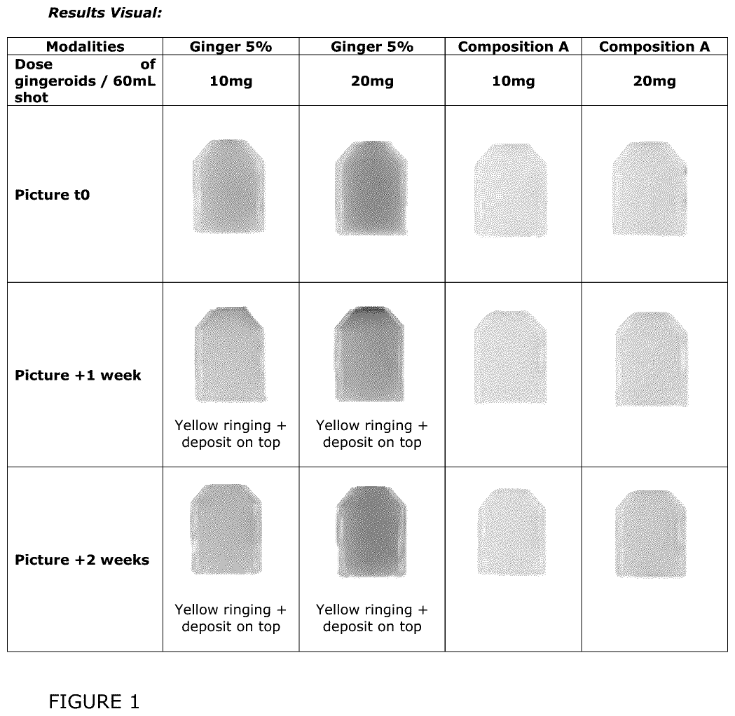

Characterization: Pictures (eyes overview, sedimentation observation), pH control (impact of the extract in the formulation), Delta Lab* (to assess color+sedimentation modifications), turbidity measurements (to assess cloudiness) After one week, a yellow ringing was observed on top of the reference sample bottles (Ginger 5%). We can see a lot of particles (powder) in suspension in the reference bottles (FIG. 1). Over two weeks, we do not observe, visually speaking, any significant color change for any sample with the Composition A formulation except for the reference (Ginger 5%). This phenomenon does not appear with composition A formulation at equivalent gingeroids dose (see FIG. 1).

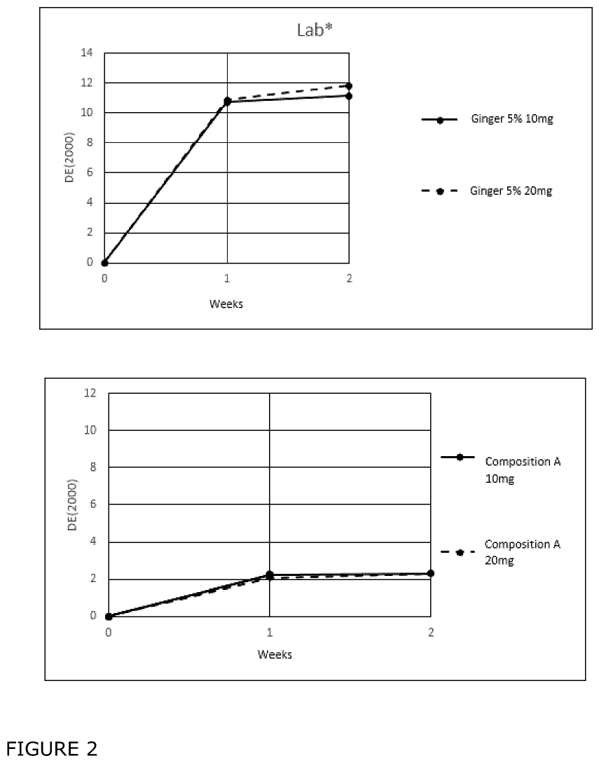

After two weeks, the reference samples (Ginger 5%) have a ΔE>10, whereas the composition A has a ΔE<3. We do not see any Lab* evolution between one and two weeks in the formulation of the invention (Composition A) (FIG. 2)

### Example 3—Simulator of the Human Intestinal Microbial Ecosystem (SHIME®)

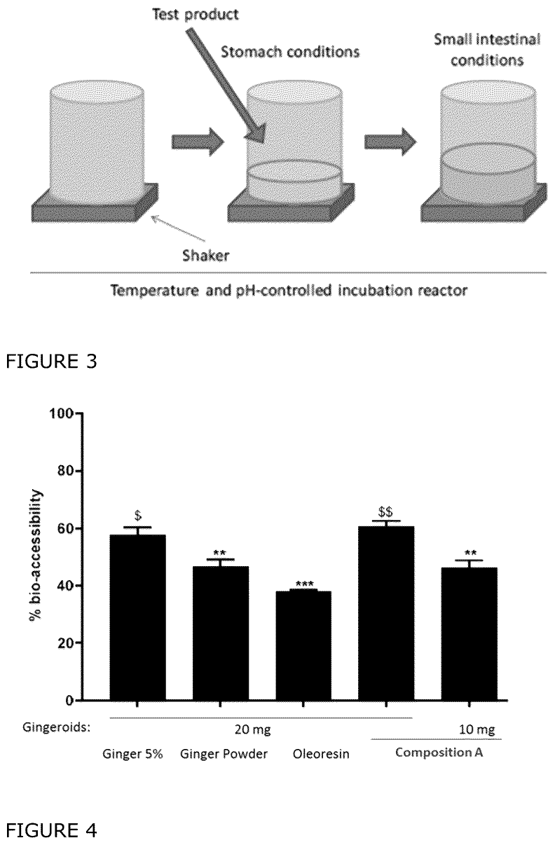

The aim of this study was to evaluate the absorption of gingeroids from 4 test Formulations at the same level of gingeroids 20 mg and 10 mg for composition A, following passage through the human upper GIT under fasted condition. For this purpose, a Simulator of the Human Intestinal Microbial Ecosystem (SHIMER) was used coupled with Caco-2 transport assays (FIG. 3).

### 3.1 Materials and Methods

The reactor setup was adapted from the SHIME, representing the gastrointestinal tract (GIT) of the adult human, as described by Molly et al. (1993)1. The SHIME consists of a succession of five reactors simulating the different parts of the human gastrointestinal tract. The first two reactors are of the fill-and-draw principle to simulate different steps in food uptake and digestion, with peristaltic pumps adding a defined amount of SHIME feed and pancreatic and bile liquid, respectively to the stomach and small intestine compartment and emptying the respective reactors after specified intervals. The last three compartments—continuously stirred reactors with constant volume and pH control—simulate the ascending, transverse and descending colon. Retention time and pH of the different vessels are chosen in order to resemble in vivo conditions in the different parts of the gastrointestinal tract.

### 3.2 Upper GIT Experiments

The typical experiment conducted by ProDigest to evaluate the bioaccessibility of actives, makes use of an adapted SHIME® system representing the physiological conditions of stomach and small intestine within the same reactor over time (FIG. 3). In order to mimic fed or fasted conditions, a specific gastric suspension is added to the reactor. After this, a standardized enzyme and bile liquid is added to simulate the small intestinal condition. Incubation conditions (pH profiles, incubation times) are optimized in order to resemble in vivo conditions in the different regions of the gastrointestinal tract for fasted or fed conditions.

In order to ensure that the simulation of the adult upper GIT under fasted and fed conditions is performed under the most representative conditions, ProDigest recently updated its in-house developed protocols based on the InfoGest consensus method—together with recent in vivo data.

Within the COST Action InfoGest, a consensus digestion method (de facto simulating a fed condition) has recently (2015) been developed with the aim to enhance comparison of digestion experiments across research teams2.

The updated ProDigest protocol however does contain such dynamic pH profiles as this mimics the in vivo condition more closely. The applied conditions are summarized below:

### Gastric Phase (Fasted State)

Incubation during 45 minutes at 37° C., while mixing via stirring, pH=2.0.
Addition of 4-fold lower pepsin and phosphatidylcholine levels4.
As the background medium, only salts and mucins are supplied.

### 3.3. Small Intestinal Phase (Fasted State)

A recent publication on the difference of human duodenal fluids in fasted and fed state conditions further required an update of the in-house developed protocols to simulate human upper GIT conditions. Based on Riethorst et al. (2015), following parameters were adapted as compared to the fed protocol:
While mixing via stirring, the pH initially automatically increases from 2.0 to 5.5 within a period of 5 minutes after which a gradually increasing pH from 5.5 to 7.0 during an incubation of 3 h at 37° C. is controlled automatically by the software (standard protocol).
Regarding pancreatic enzymes, a raw animal pancreatic extract (pancreatin) containing all the relevant enzymes in a specific ratio is used.
Regarding bile salts, 3 mM bovine bile extract is generally supplemented (bovine bile is a closer match to human than porcine in terms of tauro- and glycocholate)
Sampling at t=3 h (SI end) for coupling with Caco-2 transport assays.

### 3.4. Transport Assay in Caco-2 Cells

Caco-2 cells (HTB-37; American Type Culture Collection) were seeded on 12-well semi-permeable membranes (0.4 μm pore size) coated with collagen from rat tail (Type I) and grown for 21 days in complete medium (Dulbecco's Modified Eagle Medium (DMEM)) supplemented with 20% heatinactivated fetal bovine serum, 10 mM HEPES and 1× antibiotic-antimycotic) with 3 medium changes/week. After 21 days, the transepithelial electrical resistance (TEER) was measured to assess barrier integrity (=time point 0 h). Then, the monolayers were washed and pre-equilibrated in transport medium (HBSS+10 mM HEPES). Cells were treated apically with upper GIT medium at a concentration of 20% in transport medium. After 4 h, TEER was measured again (=time point 4 h) and the apical and basolateral fractions were collected to determine the gingeroids bioavailability. In addition, cells were lysed in PBS with 20% EtOH and 0.1% Tween-20. The apical fractions at time point 0 h were also included to determine the bioavailability. Finally, 100 μL of the apical medium upon 4 h of incubation was collected to be used in the LDH assay.

### 3.5. Test Products

The test formulations that were investigated in this phase of the project and their test doses are shown in Table 1. Formulations were added at the start of the stomach phase. All experiments were performed in biological triplicate to account for biological variability.

TABLE 6
Formulation tested

| Formulation name | % gingeroids | Dosage gingeroids (mg) |
|---|---|---|
| Ginger powder | 1.65 | 20 |
| Ginger 5% | 5.06 | 20 |
| Oleoresin | 37.30 | 20 |
| Composition A (Dose 1) | 11.83 | 20 |
| Composition A (Dose 2) | 11.09 | 10 |

TABLE 7
Composition of the formulations

| | % oleo | % quillaja | % gum | % malto | % corn starch | % silica |
|---|---|---|---|---|---|---|
| Ginger powder | 0% | 0% | 0% | 0% | 0% | 0% |
| Ginger 5% | 27% | 0% | 0% | 15% | 55% | 3% |
| Oleoresin | 100% | 0% | 0% | 0% | 0% | 0% |
| Composition A | 30% | 2% | 68% | 0% | 0% | 0% |

### 3.6. Statistics

All statistical tests were performed with the Graphpad Prism software version 8.3.0 (San Diego, USA). Statistically significant differences between treatments are shown within fasted conditions.

Statistically significant differences between gingeroids concentrations of Composition A are shown within fasted condition. Treatments are compared to composition A (20 mg) (*) or Composition A (10 mg) ($) using an ordinary one-way ANOVA with Dunnett's multiple comparisons test. (*) or ($)=p<0.05; (**) or ($$)=p<0.01; (***) or ($$$)=p<0.001 and (****) or ($$$$)=p<0.0001.

### 3.7. Results Summary.

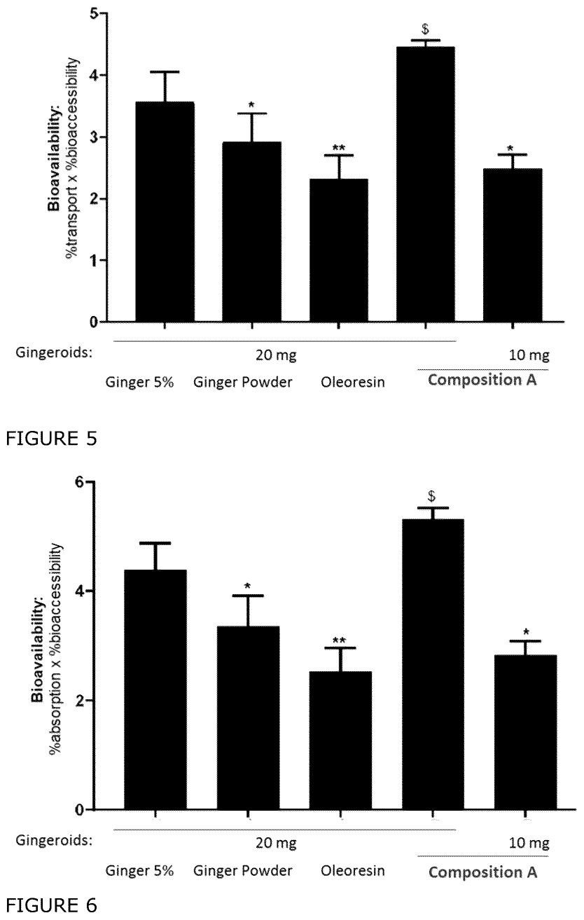

Bioaccessibility, defined as accessible gingeroids after intestinal degradation, was highest from Ginger 5% and composition A compared to ginger powder and ginger oleoresin (FIG. 4). Bioavailability, defined as bioaccessibility when taking into account transport and absorption of total gingeroids was highest from composition A compared to any other formulations (FIGS. 5 and 6).

### 3.7.1 Result 1: Bioaccessibility

The bioaccessibility of the products after passage through the upper GIT was calculated based on the measured concentrations in the apical compartment at timepoint 0 h and the theoretically added concentration of total gingeroids (i.e. ([AP 0 h]/20 or 10)*100) (FIG. 4). From this, it could be concluded that bioaccessibility of total gingeroids was highest from Composition A compared to the other formulation (FIG. 4). Moreover, bioaccessibility of total gingeroids from Composition A at a dose of 20 mg was significantly higher compared to all formulation. In contrast, bioaccessibility from Composition A at a lower dose of 10 mg was significantly not different from Oleo-resin and Ginger powder formulation at 20 mg. Composition A 10 mg was significantly lower versus Ginger 5% 20 mg but less significantly compared with Composition A 20 mg.

FIG. 4 shows the bioaccessibility of total gingeroids after passage through the upper gastro-intestinal tract (GIT). Total gingeroids concentrations present in the apical samples at time point 0 h were measured. Products were incubated under fasted upper gastro-intestinal tract (GIT) conditions at a concentration corresponding to an effective dose of either 20 mg or 10 mg gingeroids. % bioaccessibility was calculated based on the theoretical final concentration of 20 or 10 μg gingeroids/mL in the apical compartment at the start (i.e. ([AP 0 h]/20 or 10)*100). Bars represent mean±SEM (n=3). (*) represents statistically significant differences between Composition A 20 mg and the other treatments. ($) represents statistically significant differences between Composition A 10 mg and the other treatments. ($)=p<0.05; (**) or ($$)=p<0.01; (***)=p<0.001.

### 3.7.2. Result 2a and b: Bioavailability

Then, the bioavailability of total gingeroids in the Caco-2 transport assay was calculated taking into account the bioaccessibility in two different ways:

Results 2a, firstly, gingeroids bioavailability was calculated as % transport x % bioaccessibility. % transport was defined as ([BL 4 h]/20 or 10)*100, with 20 or 10 μg/mL being the theoretically added concentration of total gingeroids to the apical compartment at time point 0 h., bioavailability of total gingeroids from Composition A at a dose of 20 mg was significantly higher compared to the others formulations including Composition A 10 mg of gingeroids (FIG. 5). On the other hand, Composition A at the low dose of 10 mg was not statistically significant versus any formulations showing similar percentage of bioavailability versus the three other formulations with a twice-lower amount of gingeroid concentration at the beginning.

FIG. 5 shows the Bioavailability of total gingeroids in the Caco-2 transport assay coupled with GIT. Bioavailability is expressed as % transport x % bioaccessibility of total gingeroids to the basolateral compartment upon 4 h of incubation (i.e. % transport=([BL 4 h]/[20 or 10])*100 and % bioaccessibility ([AP 0 h]/20 or 10)*100). Products were incubated under fasted upper gastro-intestinal tract (GIT) conditions at a concentration corresponding to an effective dose of either 20 mg or 10 mg gingeroids. Bars represent mean±SEM (n=3). (*) represents statistically significant differences between Composition A 20 mg and the other treatments. ($) represents statistically significant differences between Composition A 10 mg and the other treatments. (*) or ($)=p<0.05; (**)=p<0.01.

Results 2b, secondly, gingeroids bioavailability was calculated as % absorption x % bioaccessibility. % absorption was defined as (([BL 4 h]+[cells 4 h])/20 or 10)*100, with 20 or 10 μg/mL being the theoretically added concentration of total gingeroids to the apical compartment at time point 0 h. bioavailability of total gingeroids from Composition A at a dose of 20 mg was significantly higher compared to the others formulations including Composition A 10 mg of gingeroids (FIG. 6). On the other hand, Composition A at the low dose of 10 mg was not statistically significant versus any formulations showing similar percentage of bioavailability versus the three other formulations with a twice-lower amount of gingeroid concentration at the beginning.

FIG. 6 shows the Bioavailability of total gingeroids in the Caco-2 transport assay coupled with GIT. Bioavailability is expressed as % absorption x % bioaccessibility of total gingeroids to the basolateral compartment and in the cells upon 4 h of incubation (i.e. % absorption=(([BL 4 h]+[cells 4 h])/[20 or 10])*100 and % bio-accessibility ([AP 0 h]/20 or 10)*100). Products were incubated under fasted upper gastro-intestinal tract (GIT) conditions at a concentration corresponding to an effective dose of either 20 mg or 10 mg gingeroids. Bars represent mean SEM (n=3). (*) represents statistically significant differences between Composition A 20 mg and the other treatments. ($) represents statistically significant differences between Composition A 10 mg and the other treatments. (*) or ($)=p<0.05; (**)=p<0.01.

In conclusion, at similar level of gingeroids concentration bioaccessibility and bioavailability of total gingeroids was highest for Composition A formulation compared to Ginger 5%, Ginger Powder and Oleoresin. In addition, composition A used at a lower dosage of 10 mg there was no difference of either bioaccessibility nor bioavailability compared to other formulation at 20 mg. This results demonstrate that by lowering the dosage by 2 of composition A, bioavailability and bioaccessibility are equivalent of a twice higher concentration of gingeroids within ginger 5%, ginger powder and ginger oleoresin.

### Example 4. In Vivo Study

The aim of this study was to evaluate the absorption of gingeroids metabolites including free, glucuronide and sulfate metabolites of 6, 8, 10 gingerols and of 6, 8, 10 shogaols in their free, from 3 formulations: dry ginger powder, ginger 5% and Composition A (formulation the below table).

TABLE 8
Composition of the tested formulations

| | % oleo | % quillaja | % gum | % malto | % corn starch | % silica |
|---|---|---|---|---|---|---|
| Ginger powder | 0% | 0% | 0% | 0% | 0% | 0% |
| Ginger 5% | 27% | 0% | 0% | 15% | 55% | 3% |
| Composition A | 30% | 2% | 68% | 0% | 0% | 0% |

### 4.1. Material and Method

The ginger samples were submitted to the pharmacokinetic study using vivo model involved the oral administration to mice. All animals were provided by Cell for Cell Laboratory Animal Center under the animal care protocol N° E-005-20. Pharmacokinetic studies were performed in male BALB/c (7-9 weeks, 20-25 g body weight) randomly divided into 3 groups (n=5) maintained under controlled environmental considering 12 h light and dark cycles at 22° C.

Before the experiment, food and water were provided ad libitum and all mice were acclimatized for 1 week prior to testing. Prior to the study, the mice were fasted during 1.5 hour and marked on the tail. Ginger powder was suspended in water to obtain a concentration of 2.5 mg/mL in the gavage solution. The mice were performed by intragastric administration using rigid dosing cannula at 6.3 mg/kg body weight in a single dose and tramadol (20 mg/Kg of tramadol hydrochloride at 0.5 mg/mL—Drag Pharma) was injected subcutaneously to avoid suffering during the administration. Blood samples were collected at different fixed times (0.25, 0.5, 1, 2, 4, 8 h) after gavage. At each time, ketamine/xylazine anesthesia was used as a procedure for euthanasia, and then blood samples were extracted from the celiac artery. Whole blood was collected in tube that contained EDTA and the plasma was obtained by centrifugation at 7500 rpm for 15 min at 4° C. Then the samples were stored in aliquots and frozen at −80° C. until the analysis.

### 4.2. Sample Preparation:

A calibration curve was prepared in the range 2-2500 ng/ml for each 6 gingeroids (Phytolab, Vestenbergsgreuth, Germany) adding 25 ng/ml of PAV (Perlagonic acid vanillylamide Merck, Darmstadt, Germany) as an internal standard to control retention time stability and correct system deviation. Methanol was used as the diluent for each stock standard solution. For free gingeroids determination, exactly 200 μL of internal standard solution (31.25 ng/ml in methanol) was loaded over 50 μL of plasma sample into Captiva ND 96 wells plate (Agilent #A5969002). After mixing (30 s) and filtration (under vacuum), the eluate is ready to be injected into LC/MS system. For the determination of total conjugated gingeroid metabolites (glucuronide and sulfate metabolites), 40 μL of plasma sample was mixed with 40 μL of enzyme solution (either glucuronidase 1000 units/mL, Sigma #G7017; or sulfatase, Sigma #S9626, 100 units/mL both in acetate buffer pH 5.0) for 1 hour at 37° C. After this hydrolysis step, 50 μL of the solution is mixed with 200 μL of methanol (containing internal standard) into Captiva ND 96 wells plate as well. After mixing (30 s) and filtration (under vacuum), the eluate is ready to be injected into LC/MS system.

### 4.3. LC/MS Conditions:

The liquid chromatography system used was an Agilent Infinity 1290 system coupled with an Agilent 6420 Triple quadrupole mass spectrometer in electrospray ionization mode. Autosampler was kept at 15° C. The metabolites were eluted from the Poroshell 120 EC-C8 column (50×3 mm, 1.9 μm; Agilent) at 25° C. with a mobile phase consisting of 0.1% formic acid in water LC-MS grade (solvent A) and 0.1% formic acid in acetonitrile LC-MS grade (solvent B), at a flow rate of 0.8 mL/min. The elution was as follows: 0-1 min, 45-45% B; 1-2 min, 50% B; 2-4 min, 55% B; 4-8 min, 90% B. The injection volume was 5 μL for standards and samples. For each compound, a relevant transition of the precursor-to-product ions was determined. MRM transitions were optimized using direct infusion and Optimizer B.08.00 workstation software solution (Agilent technologies, Santa Clara, CA, USA). Two qualifiers were selected to validate the specificity of the detected peak (see Table 1). The mass spectrometer parameters were set as follows: ESI source in positive mode; drying gas (N2) flow rate, 10 L/min; gas temperature, 300° C.; nebulizer, 40 psi; and capillary, 4.0 kV. The MS system fully calibrated prior to running according to manufacturer's guidelines. Datas analysis were post processed with Agilent MassHunter Quantitative/Qualitative analysis B.07.00 (Agilent technologies, Santa Clara, CA, USA).

TABLE 9
Retention times (Tr), multiple reaction monitoring (MRM) transitions,
and optimized tandem mass spectrometry (MS/MS) detection parameters
for the 6 gingeroids and internal standard.

| | | | | | | Cell | Ret | | |
|---|---|---|---|---|---|---|---|---|---|
| | | Prec | Prod | Frag | CE | Acc | Time | Ret | |
| Cpd Name | ISTD? | Ion | Ion | (V) | (V) | (V) | (min) | Window | Polarity |
| 6-gingerol | No | 277.18 | 177.1 | 90 | 8 | 7 | 1.16 | 0.5 | Positive |
| 6-gingerol | No | 277.18 | 145 | 90 | 24 | 7 | 1.16 | 0.5 | Positive |
| 6-gingerol | No | 277.18 | 117.1 | 90 | 52 | 7 | 1.16 | 0.5 | Positive |
| 8-gingerol | No | 305.21 | 177 | 130 | 8 | 7 | 2.5 | 0.5 | Positive |
| 8-gingerol | No | 305.21 | 137 | 130 | 24 | 7 | 2.5 | 0.5 | Positive |
| 8-gingerol | No | 305.21 | 115 | 130 | 68 | 7 | 2.5 | 0.5 | Positive |
| 6-shogaol | No | 277.18 | 137 | 75 | 12 | 7 | 2.86 | 0.5 | Positive |
| 6-shogaol | No | 277.18 | 122 | 75 | 40 | 7 | 2.86 | 0.5 | Positive |
| 6-shogaol | No | 277.18 | 94 | 75 | 56 | 7 | 2.86 | 0.5 | Positive |
| 10-gingerol | No | 333.25 | 177.1 | 125 | 8 | 7 | 4.32 | 0.5 | Positive |
| 10-gingerol | No | 333.25 | 145 | 125 | 28 | 7 | 4.32 | 0.5 | Positive |
| 10-gingerol | No | 333.25 | 115 | 125 | 68 | 7 | 4.32 | 0.5 | Positive |
| 8-shogaol | No | 305.21 | 137 | 70 | 8 | 7 | 4.82 | 0.5 | Positive |
| 8-shogaol | No | 305.21 | 122 | 70 | 44 | 7 | 4.82 | 0.5 | Positive |
| 8-shogaol | No | 305.21 | 94 | 70 | 60 | 7 | 4.82 | 0.5 | Positive |
| 10-shogaol | No | 333.25 | 137 | 85 | 12 | 7 | 6.21 | 0.5 | Positive |
| 10-shogaol | No | 333.25 | 122 | 85 | 64 | 7 | 6.21 | 0.5 | Positive |
| 10-shogaol | No | 333.25 | 94 | 85 | 48 | 7 | 6.21 | 0.5 | Positive |
| PAV | Yes | 294.21 | 137 | 90 | 12 | 7 | 1.67 | 0.5 | Positive |
| PAV | Yes | 294.21 | 94 | 90 | 56 | 7 | 1.67 | 0.5 | Positive |

### 4.5. Captiva ND Protocol for the Plasma Treatment Before Injection.

a) Add 50 μl of plasma* into Capitiva ND
b) Add 200 μl of Methanol with Internal Standard
c) Mix
d) Pull vacuum until all volume is though cartridge for complete elution

### 4.6. ANNEXES

### Standard and Reagents

6-gingerol assay (HPLC)≥95% (Phytolab, Ref. 89201),
8-gingerol assay (HPLC)≥95% (Phytolab, Ref. 89202),
10-gingerol assay (HPLC)≥95% (Phytolab, Ref. 89203),
6-shogaol assay (HPLC)≥90% (Phytolab, Ref. 89792),
8-shogaol assay (HPLC)≥90% (Phytolab, Ref. 83910),
10-shogaol assay (HPLC)≥90% (Phytolab, Ref. 83911),
Perlagonic acid vanillylamide PAV (internal standard) (Sigma, Ref V9130)
Beta-Glucuronidase Type HP-2 from Helix pomatia (Sigma, Ref. G7017)
Sulfatase Type H-1 from Helix pomatia (Sigma, Ref. S9626)

### Material:

Captiva ND 96-well filter plate, 0.2 μm pore size, polypropylene filter (Agilent A5969002)

### 4.7. Results

As reported in Table 10 and 12 the data demonstrated that Composition A had a significantly higher Area Under the Curve (AUC) compared with ginger powder or with ginger 5%. The AUC increased is respectively of +53.08% and +26.26%.

The higher bioavailability combined with the slow released is confirmed by the relative bioavailability data, in table 14, for which one gingeroids of composition A is 1.96 more bioavailable than one gingeroids of ginger powder and 1.38 more bioavailable than one gingeroids of ginger 5%.

In addition, the Cmax (Table 10 and 12) are not statistically different between groups (table 11 and 12, data at 30 min, p>0.05). This result indicates no statistically significant difference of the highest gingeroids level blood concentration for any groups.

On the other hand, composition A induces a statistically significant shift of the half-life (t 1/2 (h) reported in table 9 and 11) with a 2,84-fold increase compared with Ginger powder and a 1.80 fold increase compared with Ginger 5%. This shift is due to statistically significant higher gingeroids plasmatic level at 30 min, 1 h, 4 h and 8 h after ingestion of composition compared A compared with ingestion of ginger powder and at 30 min and 1 h compared with ingestion of ginger 5% (Table 10 and 11 reported values at p<0.05 in bold).

These results highlight a slow release effect of composition A demonstrated by higher gingeroids blood level from 30 min lasting up to 8 h showing a more prolonged gingeroids release in time, in other word, composition A provide a gingeroids slow release effect characterised by longer Half-life and prolonged release in time up to 8 h after ingestion.

TABLE 10
Pharmacokinetic parameters on total metabolites of
Ginger powder and Composition A.

| | PK parameters-Total | |
|---|---|---|
| | gingeroids metabolites | |
| | Ginger powder | Composition A |
| Gavage solution (mg/mL) | 314.1 | 278.9 |
| Volume of gavage- | 0.39 | 0.479 |
| average(mL) | | |
| C0 (ppb) | 23992 | 3135 |
| t 1/2 (h) | 0.07162 | 0.2038 |
| Tmax (h) | 0.25 | 0.25 |
| Cmax (ppb) | 2184 | 1417 |
| K (1/h) | 9,678 | 3.4 |
| R2 | 0.885 | 0.785 |
| AUC 0-8 (ng/ml*h) | 666.6 | 1421 |
| Δ AUC | 53.08% | |
| Increase of composition | +113% | |
| A vs. Ginger powder | | |

TABLE 11
Pharmacokinetic comparison of total gingeroids metabolites plasmatic level at any
time points between Ginger powder and Composition A (mean +/− SEM).

| | Total gingeroids metabolites plasmatic level-Mean ± SEM | | |
|---|---|---|---|
| Time (h) | Ginger powder | Composition A | p-values |
| 0.25 | 2184 ± 396.2 | 1417 ± 234.4 | 0.106 |
| 0.5 | 243.7 ± 35.86 | 670.1 ± 48.07 | <0.05 |
| 1 | 68.84 ± 14.11 | 240.5 ± 34.56 | <0.05 |
| 2 | 118 ± 33.41 | 137.4 ± 24.44 | 0.606 |
| 4 | 16.54 ± 3.47 | 112.94 ± 3.24 | <0.05 |
| 8 | 11.98 ± 7.34 | 133.5 ± 27.39 | <0.05 |

TABLE 12
Pharmacokinetic parameters on total metabolites of Ginger 5% and Composition A.

| | PK parameters-Total gingeroids metabolites | |
|---|---|---|
| | Ginger 5% | Composition A |
| Gavage solution (mg/mL) | 257.3 | 278.9 |
| Volume of gavage-average(mL) | 0.53 | 0.479 |
| C0 (ppb) | 7548 | 3135 |
| t 1/2 (h) | 0.113 | 0.2038 |
| Tmax (h) | 0.25 | 0.25 |
| Cmax (ppb) | 1670 | 1417 |
| K (1/h) | 6.228 | 3.4 |
| R2 | 0.954 | 0.785 |
| AUC 0-8 (ng/ml*h) | 1048 | 1421 |

| | |
|---|---|
| Δ AUC | 26.25% |
| Increase of composition A vs. Ginger 5% | +36% |

TABLE 13
Pharmacokinetic comparison of total gingeroids metabolites plasmatic level at any
time points between Ginger 5% and Composition A (mean +/− SEM).

| | Total gingeroids metabolites plasmatic level-Mean ± SEM | | |
|---|---|---|---|
| Time (h) | Ginger 5% | Composition A | p-values |
| 0.25 | 1767 ± 169.6 | 1417 ± 234.4 | 0.329 |
| 0.5 | 428.1 ± 39.3 | 670.1 ± 48.07 | <0.05 |
| 1 | 128 ± 9.63 | 240.5 ± 34.56 | <0.05 |
| 2 | 106.1 ± 19.08 | 137.4 ± 24.44 | 0.319 |
| 4 | 104.60 ± 24.1 | 112.94 ± 3.24 | 0.670 |
| 8 | 54.92 ± 19.64 | 133.5 ± 27.39 | 0.082 |

TABLE 14
AUC0-8h dose normalized of total gingeroids and relative bioavailability
of Composition A compared to Ginger powder or Ginger 5%

| | | Relative | Relative |
|---|---|---|---|
| | Dose-normalized AUC | Bioavailability | Bioavailability |
| | 0-8h of total gingeroid | (against Ginger | (against |
| Name | (ng*h/mL/mg) | powder) | Ginger 5%) |
| Ginger 5% | 7.68 | | |
| Ginger powder | 5.43 | | |
| Composition A | 10.64 | 1.96 | 1.38 |

This values were obtained by GraphPad software using Mean, SEM, n

### 5. Human Pharmacokinetic Study Example

### Example 5: Testing the Effect of the Composition a to Enhance the Bioavailability of Gingeroids in Healthy Volunteers Through a Comparative Pharmacokinetic Study

The aim of this study is to compare the efficacy of the composition A versus standard ginger root powder and standard ginger extract 5% in humans. The hypothesis of this study is that the Composition A is superior in terms of bioavailability in plasma as measured by the dose-normalized Area under the curve (AUC) compared to ginger root powder providing the same quantity of active substance (gingeroids).

The primary objective of the study is to assess plasmatic concentration profile of total gingeroids (6-, 8-, 10-gingerol and 6-, 8-, 10-shogaol and their glucuronide and sulfate metabolites) on a 8-hour period after consumption of a single dose of 100 mg (16.7 mg/kg body weight assuming a 60 kg human) of the Composition A compared to a single dose of ginger root powder that delivers the same amount of gingeroids.

Secondary objectives of the study are to assess plasmatic concentrations profiles of the following parameters, after consumption of a single dose of each test products:
Total gingeroids;
Parent compounds: 6-, 8-, 10-gingerol and 6-, 8-, 10-shogaol;
Metabolites: 6-, 8-, or 10-gingerol glucuronide, 6-, 8-, or 10-gingerol sulfate, 6-, 8-, or 10-shogaol glucuronide, 6-, 8-, or 10-shogaol sulfate.

Secondary objectives of the study include the assessment of the comparison between Composition A and standard ginger extracts.

The pharmacokinetic study is a monocentric, randomized, crossover, pilot and open clinical trial. There is a screening/inclusion visit (V0) followed by three experimental sessions (V1 to V3) during which studied products are consumed by subjects (one different product at each session for each randomized subject). V1 visit, take place 3 weeks maximum after V0, and can also constitute the randomization visit. Each experimental session (V1 to V3) is separated by 1 week minimum and 2 weeks maximum. During each experimental session, subjects undergo blood samples kinetic during 8 hours. The last kinetic blood sample is realized after each experimental session, 8 hours after the beginning of the kinetic. Urine collection is also performed during these visits for biobanking. Subjects have to collect their first urination the morning of each experimental visit (totality of this first urination), and 0 to 8 hour urines during the kinetic on site. The end of study can be the day after the last experimental session V3.

For the study, healthy, males and females (aged 18-35 y for instance) are recruited to take part in this study. Participants are enrolled in the study if they fulfill all inclusion criteria and present none of the exclusion criteria. Ethical approval is gained from the ethics committee. Participants are included in the study if they:
Are healthy male and female,
Are aged between 18 and 45 years old (limits included),
Are with a BMI between 19 and 25 kg/m² (limits included),
Are weight stable within +3 kg in the last three months,
Have routine blood chemistry values within the normal range
For women: are non-menopausal with the same reliable contraception since at least 3 cycles before the beginning of the study and agreeing to keep it during the entire duration of the study (condom with spermicidal gel and estrogen/progestin combination contraception accepted) or are menopausal without or with hormone replacement therapy (estrogenic replacement therapy begun from less than 3 months excluded),
Are non-smokers or have tobacco consumption ≤5 cigarettes/day and agreeing not to smoke during all experimental sessions (V1 to V3),
Agree not to consume food, drink and condiment containing ginger, ginger powder or ginger extract, for 1 week prior to and throughout the entire study,
This study is performed in cross over, each subject consumes the three tested products in a random order during each experimental session (V1 to V3). After randomization, the subjects (30 participants) consume each tested product one time, at each experimental session (V1 to V3), with a drink of water (strictly 240 ml). The tested products are consumed at TO time-point of the kinetic. Studied products are directly administered to subjects during experimental session (V1 to V3) according to random list of product attribution sequence.

Three products, which are dietary supplements in shape of capsules, are tested as part of this study:
Composition A 100 mg, with a minimum of 10% gingeroids consumed as capsules (1 capsule; 100 mg per capsule),
Standard Ginger root powder 700-800 mg, delivering the same quantity of gingeroids and consumed as capsules (2 capsules; 350-400 mg per capsule),
Standard Ginger root extract 200 mg, with a minimum of 5% gingeroids consumed as capsules (1 capsule; 200 mg per capsule).
In order to ensure about healthy status of subjects and to check eligibility criteria, a blood sample is realized during V0 visit for control record analysis and pregnancy test for non-menopausal women (βhCG dosage). Subject have to come at the clinical investigation center in a 12-hour fasting state. After realization of physical examination and verification of eligibility criteria the blood sampling is realized. Only one prick can be necessary and a maximum of 10 mL will be collected. Parameters are analyzed in serum and plasma, thus EDTA, fluor and dry tubes are used. Measurement of blood pressure are performed at each visit. It is performed during the physical examination with an electronic blood pressure monitor. Heart Rate (HR, in bpm), Systolic Blood Pressure (SBP, in mmHg) and Diastolic Blood Pressure (DBP, in mmHg) are assessed. Subjects are asked to come at the clinical investigational site in a 12-hour fasted state.
Kinetic can last 8 hours on site. Subjects stay at the clinical investigational center during all the kinetic. Ten (10) blood samplings are realized according to the following schedule:
T-5 (baseline),
T15>T30>T45>T60>T75>T90>T105>T120>T150>T180>T240>T300>T360>T480,
A margin of ±30 s is authorized for T15, ±1 min for T30 and T45, ±2 min for T60, T75, T90, T105, ±5 min for T120 to T480.

T0 time point correspond to study product consumption. Volunteer is allowed to consume his/her standard launch about 4 hours after study product consumption (just after at T240 time-point) and standard afternoon snack about 8 hours after study product consumption. Lunch will have to be consumed in 30 minutes maximum. Water is not permitted 1 h before and 1 h after product administration.

Biological parameters assessed with these samplings are analyzed in plasma thus only EDTA tubes are used (5 mL per sampling).

6-, 8-, 10-gingerol and 6-, 8-, 10-shogaol and their metabolites are analyzed using a LC/MS method as described in Example 4.

Dose-normalized Area Under the Curve (AUC) of total gingeroids (sum of 6-, 8-, 10-gingerol and 6-, 8-, 10-shogaol and their metabolites) plasmatic concentration from 0 to 8 hours (AUC0-8 h/dose) (expressed in ng·h/mL/mg) following consumption of ginger products is calculated. The dose normalized AUC is the AUC normalized according to gingeroids intake by dividing the observed AUC by the corresponding gingeroids dosage of each administration. The following time-points are considered: T15, T30, T45, T60, T75, T90, T105, T120, T150, T180, T240, T300, T360 and T480. T-5 can be considered as baseline value (T0) for AUC calculation. The primary comparison is Composition A 100 mg vs Standard Ginger root powder 700-800 mg.

The secondary endpoints following consumption of the different formulations in this research are assessed:
AUC of total gingeroids plasmatic concentration from 0 to 8 hours (AUC0-8 h) (expressed in ng·h/mL);
AUC of gingeroids separately (6-, 8-, 10-gingerol and 6-, 8-, 10-shogaol) and their metabolites plasmatic concentrations from 0 to 8 hours (AUC0-8 h) (expressed in ng·h/mL);
AUC of total gingeroids plasmatic concentration from 0 to infinity (AUC0-∞/dose) normalized according to gingeroids intake (expressed in ng·h/mL/mg);
AUC of total gingeroids plasmatic concentration from 0 to infinity (AUC0-∞) (expressed in ng·h/mL);
AUC of gingeroids separately and their metabolites plasmatic concentrations from 0 to infinity (AUC0-∞) (expressed in ng/ml·h);
Peak of total gingeroids plasmatic concentration (Cmax/dose) normalized according to gingeroids intake (expressed in ng/ml/mg);
Peak of total gingeroids plasmatic concentration (Cmax) (expressed in ng/ml);
Peak of gingeroids separately and their metabolites plasmatic concentrations (Cmax) (expressed in ng/ml);
Half-life time of total gingeroids plasmatic concentration (t1/2, expressed in minutes);
Half-life time of gingeroids separately and their metabolites (t1/2, expressed in minutes);
Terminal elimination rate constant of total gingeroids in plasma (λz);
Terminal elimination rate constant of gingeroids separately and their metabolites in plasma (λz);
Time to peak of total gingeroids plasmatic concentration (Tmax, expressed in minutes);
Time to peak of gingeroids separately and their metabolites (Tmax, expressed in minutes);
Relative bioavailability, defined as the ratio of the dose-normalized AUC0-8 h of total gingeroids for the different tested formulation to the dose-normalized AUC0-8 h obtained for the reference product (standard ginger root powder).

For AUC, following time-points are considered: T0, T15, T30, T45, T60, T90, T120, T240, T360, T480 and T24H. T-5 can be considered as baseline value (TO) for AUC calculation. Gingeroids are defined as 6-, 8-, 10-gingerol and 6-, 8-, 10-shogaol and metabolites are defined as 6-gingerol glucuronide, 6-gingerol sulfate, 8-gingerol glucuronide, 8-gingerol sulfate, 10-gingerol glucuronide, 10-gingerol sulfate, 6-shogaol glucuronide, 6-shogaol sulfate, 8-shogaol glucuronide, 8-shogaol sulfate, 10-shogaol glucuronide, 10-shogaol sulfate. Total gingeroids can correspond to sum of 6-, 8-, 10-gingerol and 6-, 8-, 10-shogaol and their metabolites in plasma.

Statistical analyses are performed on both Intent-to-treat (ITT) and Per Protocol (PP) populations.

The main analysis of bioavailability is dose-normalized AUC between 0 and 8 hours. It is analyzed with a mixed model for repeated measurements (SAS® PROC MIXED, statistical model n°1):

$$Y = Product + Visit + Baseline + {Subject}_{random}$$

With:
Y: Dose-normalized AUC between 0 and 8 hours of the analyte plasmatic concentration
Product: Standard Ginger root powder or Standard Ginger root extract 5% or Composition A 100 mg,
Visit: Visit V1 to V3
Baseline: Parameter's value at T-5 time-point (TO for AUC calculation);
Subject_{random}: Random factor.

The same statistical model is used to assess all the secondary endpoints of bioavailability evaluation.

Results show an increase bioavailability of gingeroids when consumed in the form of the Composition A in comparison to the Standard Ginger root powder.

Results show an increase bioavailability of gingeroids when consumed in the form of the Composition A in comparison to Standard Ginger root extract 5%.

1. A composition comprising:
(i) gingeroids;
(ii) a gum selected from the group consisting of gum Arabic, guar gum, xantham gum, locust bean gum, gum tragacanth and mixtures thereof; and
(iii) at least one saponin.

2. (canceled)

3. The composition according to claim 1 comprising:
(i) gingeroids;
(ii) gum Arabic; and
(iii) at least one saponin.

4. The composition according to claim 1, any one of the preceding claims, wherein the gingeroids comprise 6-gingerol 8-gingerol, 10-gingerol, 6-shogaol, 8-shogaol, 10-shogaol or mixtures thereof

5. The composition according to claim 1, wherein component (i) is a ginger oleoresin, optionally obtained via CO2 extraction.

6. The composition according to claim 5, wherein the ginger oleoresin comprises at least about 10% gingerols.

7. The composition according to claim 1, wherein the gingeroids are obtained from Zingiber officinale.

8. The composition according to claim 1, wherein the composition comprises at least about 5% gingeroids by weight of the composition.

9. The composition according to claim 1, wherein the saponin is selected from the group consisting of quillaja saponin, yucca saponin, tea saponin, liquorice saponin, gingsen saponin and mixtures thereof.

10. The composition according to claim 9, wherein the saponin is an extract obtained or obtainable from quillaja comprising more than 50% w/w of saponins.

11. The composition according to claim 1, wherein the gum is present in the composition in an amount from about 20% to about 80% by weight of the composition.

12. The composition according to claim 1, wherein the at least one saponin is present in the composition in an amount from about 0.1% to about 5% by weight of the composition (w/w).

13. The composition according to claim 1, wherein composition is in dry form and the particles have a mean diameter of from about 5 micron to about 1000 micron.

14. The composition according to claim 1, further comprising a carrier selected from the group consisting of calcium carbonate, Silica, magnesium stearate, silicate of aluminium and/or magnesium, maltodextrin, resistant maltodextrin, starch, and other soluble fibers and mixtures thereof.

15. The use of a composition according to claim 1, as nutraceutical formulation, a dietary or food product for humans or animals, a nutritional supplement, a fragrance or flavouring, a pharmaceutical or veterinary formulation, an oenological or cosmetic formulation.

16. A method of preparing a nutraceutical formulation, a dietary or food product for humans or animals, a nutritional supplement, a fragrance or flavouring, a pharmaceutical or veterinary formulation, an oenological, or cosmetic formulation, the method comprising adding the composition of claim 1 to pharmaceutically/veterinary ingredients or food acceptable ingredients as appropriate.

17. The method according to claim 15, wherein the nutraceutical formulation, dietary or food product for humans or animals, nutritional supplement, fragrance or flavouring, pharmaceutical or veterinary formulation, oenological formulation or cosmetic formulation further comprises pharmaceutically/veterinary ingredients, food acceptable ingredients and mixtures thereof as appropriate.

18. A nutraceutical formulation, a dietary or food product for humans or animals, a nutritional supplement, a fragrance or flavouring, a pharmaceutical or veterinary formulation, an oenological or cosmetic formulation consisting of, consisting essentially of or comprising a composition of claim 1.

19. The nutraceutical formulation, dietary or food product for humans or animals, nutritional supplement, fragrance or flavouring, pharmaceutical or veterinary formulation, oenological formulation or cosmetic formulation according to claim 18, further comprising pharmaceutically/veterinary ingredients, such as excipients or carriers or food acceptable ingredients and mixtures thereof as appropriate.

20. A process for the preparation of the composition of claim 1, wherein the process comprises:
(i) preparing an aqueous solution of gingeroids;
(ii) mixing the aqueous solution from (i) with an aqueous gum solution and at least one saponin and optionally a plant and/or vegetable oil to provide an emulsion; and optionally
(iii) drying the product of (ii) to provide the composition

21. A method for improving bioaccessibility, bioavailability, bio-efficacy, and/or bioactivity of gingeroids in mammals comprising the administration of said gingeroids in the form of the composition of claim 1.

22. (canceled)

23. A method for providing a slow release of gingeroids in the gut of mammals comprising the administration of said gingeroids in the form of the composition of claim 1.

24. (canceled)

25. (canceled)

26. A method of preventing or treating gastrointestinal disorders, primary dysmenorrhea, heavy menstrual bleeding, metabolic disorders, sleep disorders, neuropsychiatric disorders neurodegenerative disease, and/or aged-induced cognitive declined attention, alertness and/or mood, wherein the method comprises the administration of the composition according to claim 1.

27. (canceled)

28. The method or use according to claim 21, wherein the gingeroids comprise 6-gingerol 8-gingerol, 10-gingerol, 6-shogaol, 8-shogaol, 10-shogaol or mixtures thereof

29. The method or use according to claim 21, wherein the mammal is human.

30. The method according to claim 21, wherein the gingeroids are provided in an amount of from about 0.08 mg of gingeroids per kg body weight to about 6.5 mg of gingeroids per body weight.

31. A method for improving bioaccessibility, bioavailability, bio-efficacy, and/or bioactivity of gingeroids in mammals comprising the administration of the nutraceutical formulation, dietary or food product for humans or animals, nutritional supplement, pharmaceutical or veterinary formulation according to claim 18.

32. A method for providing a slow release of gingeroids in the gut of mammals comprising the administration of said gingeroids in the form the nutraceutical formulation, dietary or food product for humans or animals, nutritional supplement, pharmaceutical or veterinary formulation according to claim 18.

33. A method of preventing or treating gastrointestinal disorders, primary dysmenorrhea, heavy menstrual bleeding, metabolic disorders, sleep disorders, neuropsychiatric disorders, neurodegenerative disease, and/or aged-induced cognitive declined attention, alertness and/or mood, wherein the method comprises the administration of the nutraceutical formulation, dietary or food product for humans or animals, nutritional supplement, pharmaceutical or veterinary formulation according to claim 18 to a patient in need thereof.
